SEQUENCE LISTING (11 sequences)

SEQ ID NO: 1 - protein, 887 aa, Arabidopsis thaliana
MAKSSADDEELRRACEAAIEGTKQSIVMSIRVAKSRGVWGKSGKLGRQMAKPRVLALSVKSKGPRKKAFLRVMKYSSGGVLEPAKMYKLKHLSKVEVITNDPSGCTFTLGFDNLRSQSVAPPQWTMRNTDDRNRLLVCILNICKDVLGRLPKVVGIDIVEMALWAKDNTPVVTTQRSTEDGEPVAESVTESDLKVTVEKELVSQAEEEDMEALLGTYVMGIGEAEAFSERLKRELQALEAANVHAILESEPLVDEVLNGLEAATNIVDDMDEWLGIFNIKLRHMREDIESIETRNNKLEMQSVNNKALIEELDKVIERLRVPSEYAASLTGGSFDEADMLQNIEACEWLAKALRGLEVPNLDPIYANMRAVKEKRAELEKLKATFVRRASEFLRNYFASLVDFMVSDKSYFSQRGQLKRPDHADLRYKCRTYARLLQHLKGLDKNCLGPLRKAYCSSLNLLLRREAREFANELRASTKVSRNPTVWLEGSTGSSQNANTDTSAVSDAYAKMLTIFIPLLVDESSFFAHFMCFEVPALAPPGGAGNDKKSQSNNDDGNDDDDLGIMDIDETDKKPGKNSPDLTALNESLQDLLDGIQEDFYAVVDWAYKIDPLRCISMHGITERYLSGQKADAAGFVRLLLGDLESRVSMQFSRFVDEACHQIERNERNVRQMGVLPYIPRFAALATRMEQYIQGQSRDLVDQAYTKFVSIMFVTLEKIAQQDPKYADILLLENYAAFQNSLYDLANVVPTLAKFYHQASEAYEQACTRHISMIIYYQFERLFQFAKKIEDFMYTITPEEIPFQLGLSKVELRKMLKSSLSGVDKSIAAMYKKLQKNLASEELLPSLWDKCKKEFLDKYESFVQLVAKVYPSENVPGVTEMRGLLASM

SEQ ID NO: 2 - DNA, 15 bp, Artificial Sequence
  Synthetic GUS-1 primer
cgtcctgtagaaacc

SEQ ID NO: 3 - DNA, 15 bp, Artificial Sequence
  Synthetic GUS-2 primer
acgcacagttcatag

SEQ ID NO: 4 - DNA, 15 bp, Artificial Sequence
  Synthetic BIN19-1 primer
ttgctcatgttaccg

SEQ ID NO: 5 - DNA, 15 bp, Artificial Sequence
  Synthetic BIN19-2 primer
gcagttccgcaaata

SEQ ID NO: 6 - DNA, 27 bp, Artificial Sequence
  Synthetic Oligo-105 primer
gaagaattcgaacttgacgccgatacc

SEQ ID NO: 7 - DNA, 24 bp, Artificial Sequence
  Synthetic Oligo-107 primer
aggctgcagacatgcgtattttcg

SEQ ID NO: 8 - protein, 894 aa, Homo sapiens
MTAIKHALQRDIFTPNDERLLSIVNVCKAGKKKKNCFLCATVTTERPVQVKVVKVKKSDKGDFYKRQIAWALRDLAVVDAKDAIKENPEFDLHFEKIYKWVASSTAEKNAFISCIWKLNQRYLRKKIDFVNVSSQLLEESVPSGENQSVTGGDEEVVDEYQELNAREEQDIEIMMEGCEYAISNAEAFAEKLSRELQVLDGANIQSIMASEKQVNILMKLLDEALKEVDQIELKLSSYEEMLQSVKEQMDQISESNHLIHLSNTNNVKLLSEIEFLVNHMDLAKGHIKALQEGDLASSRGIEACTNAADALLQCMNVALRPGHDLLLAVKQQQQRFSDLRELFARRLASHLNNVFVQQGHDQSSTLAQHSVELTLPNHHPFHRDLLRYAKLMEWLKSTDYGKYEGLTKNYMDYLSRLYEREIKDFFEVAKIKMTGTTKESKKFATLPRKESAVKQETESLHGSSGKLTGSTSSLNKLSVQSSGNRRSQSSSLLDMGNMSASDLDVADRTKFDKIFEQVLSELEPLCLAEQDFISKFFKLQQHQSMPGTMAEAEDLDGGTLSRQHNCGTPLPVSSEKDMIRQMMIKIFRCIEPELNNLIALGDKIDSFNSLYMLVKMSHHVWTAQNVDPASFLSTTLGNVLVTVKRNFDKCISNQIRQMEEVKISKKSKVGILPFVAEFEEFAGLAESIFKNAERRGDLDKAYTKLIRGVFVNVEKVANESQKTPRDVVMMENFHHIFATLSRLKISCLEAEKKEAKQKYTDHLQSYVIYSLGQPLEKLNHFFEGVEARVAQGIREEEVSYQLAFNKQELRKVIKEYPGKEVKKGLDNLYKKVDKHLCEEENLLQVVWHSMQDEFIRQYKHFEGLIARCYPGSGVTMEFTIQDILDYCSSIAQSH

SEQ ID NO: 9 - protein, 571 aa, Rattus norvegicus
MMPVASSLIKMPGLFHPVIRPWEIPKGPEIRNTVIPNTDTTSGSFNYMDYLSRLYEREIKDFFEVAKMKMTGTTKESKKFATLPRKESAVKQETERMSQGRVSCSVSTVLSDPHRCPICFGLESTIALPSGQDPVSLHGSSGKLTGSTSSLNKLSVQSSGSRRSQSSSLLDMGNMSASDLDVADRTKFDKIFEQVLSELEPLCLAEQDFISKFFKLQQHQNLSASMAEAEDLDGGSLSRPQSSGSLLPVSSEKDMIRQMMIKIFRCIEPELNNLIALGDKVDSFNSLYMLVKMSHHVWTAQNVDPASFLSTTLGNVLVTVKRNFDKCISNQIRQMEEVKISKKSKVGILPFVAEFEEFAGLAESIFKSAERRGDLDKAYTKLIRGVFINVEKVANESQKTPRDVVMMENFHHIFATLSRLKISCLEAEKKEAKQKYTDHLQSYVIYSLGQPLEKLNHFFEGVEARVAQGIREEEVSYQLAFNKQELRKVIKEYPGKEVKKGLDNLYKKVDKHLCEEENLLQVVWHSMQDEFIRQYKHFEGLIARCYPGSGVTMEFTIQDILDYCSSIAQSH

SEQ ID NO: 10 - protein, 894 aa, Mus musculus
MTAIKHALQRDIFTPNDERLLSIVNVCKAGKKKKNCFLCATVTTERPVQVKVVKVKKSDKGDFYKRQIAWALRDLAVVDAKDAIKENPEFDLHFEKVYKWVASSTAEKNAFISCIWKLNQRYLRKKIDFVNVSSQLLEESVPSGENQSVAGGDEEAVDEYQELNAREEQDIEIMMEGCECAISNAEAFAEKLSRELQVLDGANIQSIMASEKQVNTLMQLLDEALTEVDQIELKLSSYEEMLQSVKEQMDQISESNHLIHLSNTNNVKLLSEIEFLVNHMDLAKGHIKALQEGDLVSSRGIEACTNAADALLQCMNVALRPGHDMLLAIKQQQQRFSDLREHFARRLASHLNNVFVQQGHDQSSTLAQHSVELTLPNHHPFHRDLLRYAKLMEWLKSTDYGKYEGLTKNYMDYLSRLYEREIKDFFEVAKMKMTGTSKESKKFATLPRKESAVKQETESLHGSSGKLTGSTSSLNKLSVQSSGSRRSQSSSLLDMGNMSASDLDVADRTKFDKIFEQVLSELEPLCLAEQDFISKFFKLQQHQNMSASMTEAEDLDGGSLSRQHSSGTLLPVSSEKDMIRQMMIKIFRCIEPELNNLIALGDKVDSFNSLYMLVKMSHHVWTAQNVDPASFLSTTLGNVLVTVKRNFDKCISNQIRQMEEVKISKKSKVGILPFVAEFEEFAGLAESIFKNAERRGDLDKAYTKLIRGVFINVEKVANESQKTPRDVVMMENFHHIFATLSRLKISCLEAEKKEAKQKYTDHLQSYVIYSLGQPLEKLNHFFEGVEARVAQGIREEEVSYQLAFNKQELRKVIKEYPGKEVKKGLDNLYKKVDKHLCEEENLLQVVWHSMQDEFIRQYKHFEGLIARCYPGSGVTMEFTIQDILDYCSSIAQSH

SEQ ID NO: 11 - protein, 889 aa, Drosophila melanogaster
MLSIGAMANIKHTLQKELFLASGERLLSVVTVVKKKDKKPCYLCVVTTAPPVPVVTLCLIKQSEQREGEYKRKRSWQLDEIKWVDGRNEQFQTHEFDLQLEKLYKWYALNPHERQNFLAVLNRQIQKSVRGQRAEFRNVPAAWLSEKSPEKVALGRAVQKTQHMDDEEDEEEEAQEFTALTDKEANELGKLFSECDFAIKDAEQFIEQLSRELHDLDGANMQSVLASEQKVLKMMEHIDNAISEADKFENRLDSYEDILGHVKETMEKIGGKNAMIEIANNNNIKLMKELNKVISQLDLPHSQQQALDEPDLKTANGRKAAIAAAQCLQQAMNSDIDPALLRLEAVQDQRKRFEKWKQKFSATVSRFMNNLFIHLGNEIGDMQVTSTELTLPNHSNVHRELTPYTELMHWTKAMDRKTYDGLMRVYTASLSKIYDRDVRNFFNLAKIQVTEKLRNSREDLDMSTSSRKSAVSTIPYGTLGINRDQWGPGVETADRMRFDALLEKVLAELEPIALQEQLFCINFFQMDVISPTTKNTQTTLEMEKAVDMTQSIISGAVSPSGDGVPQKRIDRQINEDVRKLMMGLFGCLEPELVSFIQSFERVDSFYSLYVFVRLTQHVMSAQDTHSFLSMTFASALVQVKRSFDRFMQNQLLSIREAKLHKRSKAILPYVENFENFAQTAEGIFRKSDRRTDMEKWYLQLVNAIFEGIQLHSQEHPKTPIQVVRMENYHHMYALLAQLKVPGLDALKKEAKKCYNDALKAYVTQYFGRPLEKLNQFFEGVQLKVAQGVKETEISYQMAFSKQELRKVIAQYPAREVKKGLENLYKKVEKHLSEEENLLQVVWHAMQEEFIAQYNYLEERIQKCYAGAMINLEFNIQDILAFFSDIARSH

(19) United States
(12) Patent Application Publication
(10) Pub. No.: US 2006/0228704 A1
(43) Pub. Date: Oct. 12, 2006

(54) Visualization of introduced dna (void) in transit by in situ hybridization

(76) Inventor: Shen Quan Pan, Singpore (SG)

Correspondence Address: BIRCH STEWART KOLASCH & BIRCH, PO BOX 747, FALLS CHURCH, VA, 22040-0747, US

(21) Appl. No.: 10/510,034

(22) PCT Filed: Apr. 1, 2003
(86) PCT No.: PCT/SG03/00067

§ 371 Date: Oct. 1, 2004

Related U.S. Application Data
(60) Provisional application No. 60/368,524, filed on Apr. 1, 2002.

(51) Int. Cl. C12Q 1/68

(52) U.S. Cl. 435/6

(57) ABSTRACT

The invention relates to a process for monitoring exogenous nucleic acid in transit, termed Visualization Of Introduced DNA (VOID). Once the nucleic acid has been introduced into a cell in a biological sample, the cells are fixed and permeabilized if necessary, then subjected to an in situ hybridization procedure in which the fixed cells are contacted with a probe which hybridizes to the exogenous nucleic acid. The exogenous nucleic acid, in transit, can thus be visualized. VOID may be used (a) to determine the efficiency of delivery of the nucleic acid into the nucleus; (b) to assess the risk associated with DNA delivery procedures where, by tracking the fate of the DNA, it can be determined whether too many of the exogenous DNA are delivered, which may lead to an undesired consequence; (c) to control the copy number of DNA delivery during the development of a transgenic product or a product containing an exogenous nucleic acid as the active ingredient; (d) to identify cells as having been transformed or transfected with an exogenous DNA, without the use of selection markers or reporters; (e) to identify molecular markers associated with transformation competency of a cell and identifying a cell that is competent to receive exogenous nucleic acid; (f) identifying, characterizing and producing cells competent to receive exogenous nucleic acid. VOID has been used successfully to identify the cellular protein VirD2-Interacting protein (VDI) as such a molecular marker. Thus a cell may be identified as being transformation-competent if and when it expresses VDI.

### REFERENCE TO RELATED APPLICATIONS

This application claims the benefit of U.S. Provisional Application No. 60/368,524, filed Apr. 1, 2002, the content of which is herein incorporated by reference.

### FIELD OF INVENTION

The invention relates to a process for monitoring exogenous nucleic acid by in situ hybridization. The nucleic acid is monitored in transit once it has been introduced into a cell.

### BACKGROUND OF THE INVENTION

Recombinant DNA technology can be used for various applications in the biomedical, agricultural, environmental, and industrial fields. These often require gene or DNA delivery and transformation. DNA molecules can be delivered into mammals as DNA vaccines. DNA molecules containing useful genes can be applied as therapeutics or so-called gene therapy. Genetic modifications of animal, plant or microbial organisms based on transgenic technology can lead to development of various high value products.

To develop these gene-based or DNA-based products, it is crucial to monitor the gene (DNA) delivery and transformation processes and efficiencies. Current techniques for this purpose are based on detection of the expressed gene product of the DNA delivered. Therefore, marker or reporter genes are used for the detection of gene delivery and transformation. To allow the marker or reporter gene to be expressed, appropriate promoters that can drive the expression of the delivered gene have to be included. However, a promoter may not be functional in certain cell type(s), stage(s), and/or condition(s). The activity of a promoter in certain cell type(s), stage(s), and/or condition(s) may be too weak for the detection. Alternatively, the activity may be so strong that it can lead to the toxicity of a marker or reporter gene. In addition, enough time has to be given to the marker or reporter gene to be expressed. This can considerably prolong the time needed to develop DNA-based or gene-based products.

Inclusion of the marker or reporter gene(s) in a gene-based or DNA-based product may also harm public acceptance of the product, because of the potential effects of the marker or reporter gene(s) or its product(s) on the environment and human health. Furthermore, detection of the marker or reporter protein cannot readily reveal the transgene copy numbers or locations of the desired DNA, which are important for assessing the efficacy and safety of the material containing the desired DNA. This is because having too many copies of the transgene delivered into the target cells may lead to unwanted integration at vital sites of the host chromosomes, which could be harmful to the host.

In situ hybridization is a technique which uses direct hybridization of a DNA probe with DNA or RNA in biological structures, typically permeabilized cells, subcellular fractions, or fixed chromosome preparations. The technique is often directed toward a target sequence in a double-stranded duplex nucleic acid, typically a DNA duplex associated with a pathogen or with a selected sequence in viral or cell chromosomal DNA. A single-stranded labeled probe is annealed to the denatured target duplex nucleic acid, and the structure is processed for visualization of the annealed probe, thus allowing the location of the probe within the target duplex nucleic acid to be determined.

In situ hybridization has been used to reveal morphological information about the localization of sequence-specific targets in fixed biological structure. Specifically the method has been widely applied to chromosomal DNA, for mapping the location of specific gene sequences, and distances between known gene sequences, for studying chromosomal distribution of satellite or repeated DNA, for examining nuclear organization, for analyzing chromosomal aberrations, for localizing DNA damage in single cells or tissue and for determining chromosome content by flow cytometric analysis. Localization of integrated viral sequences within host-cell chromosomes have been reported. The method has also been used to study the position of chromosomes, by three-dimensional reconstruction of sectioned nuclei, and by double in situ hybridization with mercurated and biotinylated probes, using digital image analysis to study interphase chromosome topography. In situ hybridization has also been used to detect the presence of virus in host cells, as a diagnostic tool.

Cheng L, Bucana C D, Wei Q. Fluorescence in situ hybridization method for measuring transfection efficiency. Biotechniques September 1996; 21(3):486-91, states “We describe here the use of fluorescence in situ hybridization (FISH) to measure the transfection efficiency of the transient expression vector pCMVcat in lymphoblasts and fibroblasts.” Cheng et al. showed that, for transfection of pCMVcat by the diethylaminoethyl-dextran method, the transfection efficiency was about 15 and 70 times greater in fibroblasts and lymphoblasts, respectively, when measured by FISH as compared to the efficiency measured by cotransfection with pCMV beta gal.

In Co D O, Borowski A H, Leung J D, van der Kaa J, Hengst S, Platenburg G J, Pieper F R, Perez C F, Jirik F R, Drayer J I. Generation of transgenic mice and germline transmission of a mammalian artificial chromosome introduced into embryos by pronuclear microinjection. Chromosome Res 2000;8(3):183-91, transgenic mice were generated by pronuclear microinjection of a murine satellite DNA-based artificial chromosome (SATAC). FISH analyses of metaphase chromosomes from mitogen-activated peripheral blood lymphocytes from both the founder and progeny revealed that the SATAC was maintained as a discrete chromosome and that it had not integrated into an endogenous chromosome.

Collas P, Alestrom P. Nuclear localization signals enhance germline transmission of a transgene in zebrafish. Transgenic Res Jul. 1998;7(4):303-9 reported that cytoplasmic injection into zebrafish eggs of plasmid DNA complexed to nuclear localization signal (NLS) peptides increased nuclear uptake of transgene DNA early during embryo development. It states that “Monitoring the dynamics of nuclear uptake of DNA-NLS complexes by fluorescence in situ hybridization (FISH) of interphase nuclei indicates that NLS enhances both the proportion of nuclei importing DNA during early embryo development, and the amount of DNA imported by individual nuclei.”

### SUMMARY OF THE INVENTION

The invention relates to the use of a modified in situ hybridization procedure to monitor the progress of introduced nucleic acid as it makes its way into the cell, through the cytoplasm and into the nucleus. In the past, in situ hybridization has been used to visualize introduced DNA at its endpoint; for example, DNA after it has integrated into a chromosome, or episomal DNA once it has reached the point where it has become replicated and/or has reached the nucleus and can be expressed (transcribed). The present invention relates to visualizing the nucleic acid in transit, prior to, or just as it reaches, its endpoint. The process is useful for defining the optimum parameters for nucleic acid delivery under a given set of conditions.

The process is termed Visualization Of Introduced DNA (VOID). Once the nucleic acid has been introduced into a cell in a biological sample, the cells are fixed and optionally permeabilized if necessary, then subjected to an in situ hybridization procedure in which the fixed cells are contacted with a probe which hybridizes to the exogenous nucleic acid. The exogenous nucleic acid, in transit, can thus be visualized.

Although the abbreviation “VOID” refers to DNA, the process is clearly applicable to any introduced nucleic acid including RNA.

In one embodiment, the nucleic acid is DNA. In another embodiment, the nucleic acid is DNA and is introduced into the cell by Agrobacterium.

In situ hybridization may be fluorescence in situ hybridization, radioactive in situ hybridization, or enzymatic in situ hybridization.

In one aspect, the process described above may be used to determine the number of exogenous nucleic acid in the cytoplasm or in the nucleus.

In another aspect, the process described above may be used to determine whether the exogenous nucleic acid is in the cytoplasm or the nucleus.

In another aspect, the process described above may be used to determine the length of time required for the exogenous nucleic acid to appear in the cytoplasm.

In another aspect, the process described above may be used to determine how long it takes for the exogenous nucleic acid to reach the nucleus from the cytoplasm.

In another aspect, the process described above may be used to determine the efficiency of delivery of the nucleic acid into the nucleus. This process would further comprise the step of measuring the ratio of the number of the exogenous nucleic acid in the nucleus to the number of the exogenous nucleic acid in the cytoplasm.

In another aspect, the process described above may be used to assess risk associated with introduction of the exogenous nucleic acid into the cell, including the risk associated with the use of a particular vehicle for nucleic acid delivery such as a particular vector system. This process further comprises the step of determining the number of exogenous nucleic acid in the cytoplasm and in the nucleus at different time intervals after the exogenous nucleic acid has been introduced. The ratio of exogenous nucleic acid in the nucleus to cytoplasm is determined at each interval. This allows VOID to predict, in accordance with said ratio and number of exogenous nucleic acid introduced, the risk associated with introduction of the exogenous nucleic acid into the cell or with the use of a particular vehicle for nucleic acid delivery.

In another aspect, the process described above may be used to control the copy number of the exogenous nucleic acid introduced into the cell.

In another aspect, there is described a process for determining the proportion of cells competent to receive exogenous nucleic acid. The process comprises: (a) introducing an exogenous nucleic acid to a portion of a population of cells; (b) monitoring the exogenous nucleic acid according to the process described above to determine the presence of the exogenous nucleic acid in the cell; and (c) determining the number of cells in which the exogenous nucleic acid is present. The proportion of cells which contain the exogenous nucleic acid as observed with VOID reflects the proportion of cells competent to receive the exogenous nucleic acid.

In another aspect, VOID may be used to identify whether a cell contains an exogenous nucleic acid, without having to use a selection marker or reporter protein. The process comprises:(a) introducing the exogenous nucleic acid into the cell; and (b) monitoring the exogenous nucleic acid according to the process described above. Visualization of the nucleic acid in the cell indicates that the cell contains the exogenous nucleic acid.

In another aspect, VOID may be used to identify a molecular marker associated with the competency of a cell to receive exogenous nucleic acid. The process comprises:(a) introducing an exogenous nucleic acid to the cell; (b) monitoring the exogenous nucleic acid according to the process described above;(c) testing the fixed cells for binding of a cellular antigen with an antibody. The antibody should be capable of binding to the antigen in the fixed and permeabilized cell; and (d) determining whether the antigen co-localizes with the exogenous nucleic acid in transit. Co-localization of the exogenous nucleic acid in transit with the antigen would indicate that the antigen is a molecular marker associated with transformation competency.

In another aspect, VOID may be used to determine the optimum parameters for obtaining a desired copy number of exogenous nucleic acid introduced into the cell, the process comprising: (a) introducing an exogenous nucleic acid into a cell under a set of parameters; (b) monitoring the exogenous nucleic acid according to the process described above to determine the number of exogenous nucleic acid in the cytoplasm or in the nucleus at different time intervals after the nucleic acid has been introduced; and (c) determining the set of parameters under which the exogenous nucleic acid is delivered in the desired copy number into the cell. In one embodiment, at least one of the parameters is the length of time in which the exogenous nucleic acid is in contact with the cell.

In another aspect, VOID may be used to identify a cell that is competent for receiving exogenous nucleic acid, the process comprising monitoring the exogenous nucleic acid according to the process described above for presence of the exogenous nucleic acid in the cell.

In another aspect, VOID is used to identify a cell competent to receive exogenous nucleic acid. The process comprises identifying expression of a Sec3 protein in the cell. Sec3 expression would indicate that the cell is competent to receive exogenous nucleic acid. In one embodiment, the cell is a plant cell and the Sec3 protein is VirD2-Interacting protein (VDI).

In another aspect, there is described a process for producing cells competent to receive exogenous nucleic acid, the process comprising the step of expressing Sec3 protein in the cell under control of an inducible promoter.

In another aspect, VOID is used to identify a cell competent to receive exogenous nucleic acid. The process comprises the step of identifying expression of a component of Exocyst complex in the cell. Expression of the component indicates that the cell is competent to receive exogenous nucleic acid.

In another aspect there is described a kit for monitoring exogenous nucleic acid in transit, the nucleic acid having been introduced into a cell. The kit comprises: (a) reagents for fixing the cells; (b) reagents for permeabilizing the fixed cells; (c) reagents for in situ hybridization of a probe with the exogenous nucleic acid; and (d) instructions for using the reagents (a) to (c) to monitor the exogenous nucleic acid in transit.

In one embodiment, the processes described above are applied to plant cells. Such processes may further comprise the step of removing the cell wall.

### DESCRIPTION OF THE DRAWINGS

Various embodiments of the invention are illustrated by way of the figures described below. The figures are originally in color. The various symbols are used to indicate the colored objects observed in figures which are not printed in color.

FIG. 1. Schematic presentation of relevant gene constructs in A. tumefaciens. The Ti plasmid contains the vir genes (virA-E and virG) that are required for the transfer of T-DNA harbored on pIG121-Hm (Ohta S. et al. 1990. Construction and expression in tobacco of a β-Glucuronidase (GUS) reporter gene containing an intron within the coding sequence. Plant Cell Physiol., 31(6), 805-813) and delineated by the left (LB) and right border (RB). The T-DNA contains the GUS gene driven by the 35S promoter (P35s). The small arrows indicate the primers used to amplify PCR fragments, which were labeled as probes for VOID. Primers GUS-1 (5′-CGTCCTGTAGAAACC-3′; SEQ ID NO:2) and GUS-2 (5′-ACGCACAGTTCATAG-3′; SEQ ID NO:3) were used to generate a 755-bp fragment, which can be used as the T-DNA probe to detect T-DNA inside plant cells. Primers BIN19-1 (5′-TTGCTCATGTTACCG-3′; SEQ ID NO:4) and BIN19-2 (5′-GCAGTTCCGCAAATA-3′; SEQ ID NO:5) were used to generate a 757-bp fragment, which can be used as the vector backbone probe to detect the presence of the plasmid backbone. Primers Oligo-105 (5′-GAAGAATTCGAACTTGACGCCGATACC-3′; SEQ ID NO:6) and Oligo-107 (5′-AGGCTGCAGACATGCGTATTTTCG-3′; SEQ ID NO:7) were used to generate a 677-bp fragment, which can be used as the aopB probe to detect the presence of A. tumefaciens chromosomal DNA.

FIG. 2. The requirements for visualization of T-DNA inside plant cells. The BY2 cells were cocultivated with LBA4404(pIG121-Hm) (vir⁺) (panels A, D and F), MX243(pIG121-Hm) (virB⁻) (panel B), WR1715(pIG121-Hm) (virD2⁻) (panel C), or MX358(pIG121-Hm) (virE2⁻) (panel E) for 1 day. They were then subjected to the VOID procedure using the T-DNA (panels A, B, C and E), aopB (panel D) or vector backbone (panel F) probes (FIG. 1). Confocal microscopy was conducted to reveal specific DNA hybridization, which generated the red dots (arrowed). The green fluorescence (arrowhead) indicated BY2 nuclei stained with PicoGreen. Red fluorescence, green fluorescence and transmission images were overlapped for each panel.

FIG. 3. Visualization of plant nuclear DNA molecules and numbers of T-DNA molecules inside plant cells. The BY2 cells were cocultivated with LBA4404(pIG121-Hm) for 1 d and subjected to the VOID procedure using the plant DNA [which was a 904-bp EcoRI fragment of an Arabidopsis thaliana cDNA clone (corresponding to F16N3.18 of the genome sequence)] (panels A and B) or T-DNA (panel C) probe (FIG. 1). Confocal microscopy was conducted to reveal specific DNA hybridization, which generated the red dots (arrowed). The green fluorescence (arrowhead) indicated BY2 nuclei stained with PicoGreen. Red and green fluorescence images were overlapped (panels A and C). To clearly reveal red dots, only red fluorescence image was shown in panel B. To count numbers of T-DNA molecules inside BY2 cells, individual BY2 cells were reconstituted by overlapping sequential 1-μm-laser-sections of confocal microscopy (panel C); the white bar denotes 10 μm.

FIG. 4. Time course of T-DNA trafficking inside plant cells. The BY2 cells were cocultivated with LBA4404(pIG121-Hm) for 0 h (panel A), 2 h (panel B), 5 h (panel C), 1 d (panel D), 2 d (panel E), or 3 d (panel F). They were then subjected to the VOID procedure using the T-DNA probe (FIG. 1). Confocal microscopy was conducted to reveal specific DNA hybridization, which generated the red dots (arrowed). The green fluorescence (arrowhead) indicated BY2 nuclei stained with PicoGreen. Red fluorescence, green fluorescence and transmission images were overlapped for each panel.

FIG. 5. Localization of VDI protein (SEQ ID NO:l) in tobacco BY2 and Arabidopsis thaliana cells. Tobacco BY2 cells (A) and A. thaliana (B) cells were fixed and then subjected to the immunohistology using anti-VDI as the primary antibody and anti-rabbit IgG-Cy3 as the secondary antibody. Confocal microscopy was conducted to reveal the localization of VDI, which generated the red dots (arrowed). The green fluorescence (arrowhead) indicated BY2 nuclei stained with PicoGreen. Red fluorescence, green fluorescence and transmission images were overlapped for each panel. There were not any significant signals detectable in the negative control (C) when preimmune serum instead of anti-VDI was used. The VDI was located in the cytoplasm of cells and only existed in some cells.

FIG. 6. GUS staining of tobacco BY2 cells. Tobacco BY2 cells were cocultivated with (A) or without (B) preinduced LBA4404(pIG121-Hm) for 3 days and were then subjected to the GUS assay. Samples were then viewed under light microscopy. The blue color spots (arrowhead) represented the GUS activity, which indicated that clusters of cells were transformed by A. tumefaciens. Only some plant cells were transformed by A. tumefaciens and gave blue color spots. Bar represents 126 μm.

FIG. 7. Coexistence of VDI and GUS protein in the same transformed BY2 cells. Tobacco BY2 cells were cocultivated with preinduced LBA4404(pIG121-Hm) (A) or MX243(pIG121-Hm) (B) for 3 days. They were then subjected to the double immunohistology assay. Confocal microscopy was conducted to reveal the location of VDI and GUS protein, which generated the red dots (arrowed) and green dots (arrowhead), respectively. Red fluorescence, green fluorescence and transmission images were overlapped for each panel. Coexistence of VDI and GUS in the same transformed BY2 cells indicated that only those cells producing VDI protein could be transformed by A. tumefaciens.

FIG. 8. Colocalization of VDI protein and T-DNA molecules in the same transformed BY2 cells. Tobacco BY2 cells were cocultivated with LBA4404(pIG121-Hm) (A) or MX243(pIG121-Hm) (B) for 1 day. They were then subjected to VOID followed by immunohistology. Confocal microscopy was conducted to reveal the location of VDI and T-DNA, which generated the red dots (arrowed) and green dots (arrowhead), respectively. Red fluorescence, green fluorescence and transmission images were overlapped for each panel. Colocalization of VDI and T-DNA in the same transformed BY2 cells indicated that only those cells producing VDI protein could receive T-DNA delivered by Agrobacterium.

FIG. 9: Sequence homology between VirD2-Interacting protein (VDI; SEQ ID NO:l) and various members of the Sec3 family, including human Sec3 (SEQ ID NO:8), rat Sec3 (SEQ ID NO:9), mouse Sec3 (SEQ ID NO:10), and fruit fly Sec3 (SEQ ID NO:11).

### DETAILED DESCRIPTION OF THE EMBODIMENTS

The invention is applicable to the monitoring of any exogenous nucleic acid in transit in a cell-containing biological sample.

The nucleic acid may be any DNA or RNA which has been introduced into the cell. By “exogenous nucleic acid” is meant any nucleic acid which is not already in the cell. The term encompasses nucleic acids such as plasmid constructs, viral nucleic acids, episomal DNA and cassettes, artificial chromosomes and naked DNA, which contain a sequence which is not already present in the cell. It is contemplated that the invention also applies to the monitoring of nuclear fusion, where the DNA of one nucleus is distinguishable in sequence from the DNA of the other nucleus.

The exogenous nucleic acid is the target for in situ hybridization and may be any introduced DNA target, or may be an introduced RNA such as the genome of RNA viruses or RNA introduced by a retrovirus. The target may also be a nucleic acid which has been amplified by means such as the polymerase chain reaction (PCR), so that additional copies of the nucleic acid targets are produced.

The term “biological sample” includes, but is not limited to, samples of human, animal, microbial or plant origin such as human, animal, microbial or plant tissue sections, cell or tissue cultures, suspension of human, animal or plant cells or isolated parts thereof, human or animal biopsies, blood samples, cell-containing fluids and secretion. The introduced nucleic acids in whole cells contained in the biological sample are observed, not subcellular fractions.

Visualization of Introduced DNA (VOID)

The invention relates to the monitoring of nucleic acid in transit, after it has been introduced into the cell and as it progresses toward its endpoint. The nucleic acid is visualized while it is in flux, before or just as it reaches its endpoint. The endpoint is usually when the introduced nucleic acid is in the nucleus and becomes integrated into or associated with the host cell chromosomes, or engages the transcription machinery for expression, or engages the cellular mechanism for self-replication. While it is understood that no living cell is actually static at any point, by “in transit” is meant the progression of the introduced nucleic acid towards its equilibrium or stable state at its endpoint location in the cell. Usually this endpoint location is the nucleus of the cell into which the nucleic acid has been introduced.

The exogenous nucleic acid may be introduced into the cell by any means known in the art. The term “introduce” is sometimes used interchangeably in the art with “transform” or “transfect”. Methods for transforming/transfecting host cells with expression vectors are well-known in the art and depend on the host system selected as described in Ausubel et al., (Ausubel et al., Current Protocols in Molecular Biology, John Wiley & Sons Inc., 1994). Means for introducing exogenous nucleic acids into cells include, but are not limited to, electroporation (see for example U.S. Pat. No. 5,273,525); liposome-mediated and lipoprotein mediated delivery (see for example U.S. Pat. No. 6,468,798); viral delivery systems such as retroviral vectors (see for example U.S. Pat. No. 6,410,313), adeno associated viral vectors (see for example U.S. Pat. No. 6,153,436), herpes simplex viral vectors, and bacteriophage vectors (see for example U.S. Pat. No. 6,054,312); cationic peptide mediated delivery (see for example U.S. Pat. 6,387,700); microneedle injection (see for example U.S. Pat. No. 5,697,901); microparticle bombardment and Agrobacterium-mediated gene delivery, especially in plants (see for example U.S. Pat. No. 5,932,782); and microinjection into the nucleus (see for example U.S. Pat. No. 6,498,285). The nucleic acid may be introduced first into the cytoplasm through the plasma membrane, or directly into the nucleus through the nuclear membrane. In a preferred embodiment, the nucleic acid is introduced into the cytoplasm through the plasma membrane.

Although the actual experiments described here relate to Agrobacterium-mediated transformation of tobacco cells, VOID should be applicable to all eukaryotic cells, such as plant, yeast, fungal and animal cells, including human cells, using any kind of nucleic delivery method known in the art and as described above.

In one embodiment, DNA is introduced into the target cells by Agrobacterium-mediated transformation. In nature, A. tumefaciens is a soil-borne bacterium that causes crown gall tumours on many plant species, particularly dicot plants. The bacterium transfers a specific segment (T-DNA) of its tumour-inducing (Ti) plasmid into plant cells, where the T-DNA becomes integrated into the plant genome (Christie, P. J., 2001. Mol. Microbiol., 40(2), 294-305; Gelvin, S. B., 2000. Annu. Rev. Plant Physiol. Plant Mol. Biol., 51, 223-256; Zhu et al. 2000. J. Bacteriol., 182, 3885-3895; Zupan et al. 2000. Plant J., 23, 11-28). The T-DNA contains the oncogenes that cause overproduction of plant hormones and hence tumours. Therefore, Agrobacterium is a natural genetic engineer that transforms plants with its own genes for its own benefits.

The virulence (vir) genes located on the Ti plasmid are directly responsible for the T-DNA transfer process. The Agrobacterium genome has been sequenced. Transformation using Agrobacterium has been modified such that useful genes can be introduced into many plant species without causing tumours. Recently, it has been demonstrated that Agrobacterium can also transfer DNA into yeast, fungal and mammalian cells (P. Bundock et al. EMBO J. 14:3206 (1995); K. L. Piers et al. Proc. Natl. Acad. Sci. USA. 93:1613 (1996); M. J. de Groot et al. Nat. Biotechnol. 16:839 (1998); T. Kunik et al. Proc. Natl. Acad. Sci. U.S.A. 98:1871 (2001)). This suggests that this system can be adopted for other eukaryotic cells.

The Agrobacterium system has several features that make it very attractive as a general gene transfer vector. As the integration occurs at fairly random positions, the T-DNA can be used as a tagging vector. The T-DNA can be also targeted to a specific site in the genome by homologous recombination. Due to the accompanying VirD2 and VirE2 proteins, the T-DNA is well preserved during its passage to the nucleus. The transformation is highly efficient due to the nuclear targeting by the nuclear localization signals of VirD2 and VirE2. These nuclear localization signals are well conserved among eukaryotic cells.

VirD2 is an Agrobacterium virulence gene encoded protein that plays multiple important roles in the transfer of T-DNA. First, VirD2 serves as an endonuclease that cleaves the bacterial T-DNA at the border sequences. After cleavage, the VirD2 protein remains covalently attached to the 5′ end of the T-strand. This would enable the VirD2 protein to serve as a pilot protein that guides the passage of the T-strand from Agrobacterium into plant cells.

There are a number of plant proteins that specifically interact with VirD2. These proteins are well conserved in the eukaryotic cells, including plant, yeast, fungal and animals cells. Since T-DNA can be delivered by the bacterium into plant, yeast, fungal and mammalian cell nuclei, this suggests that the DNA trafficking pathway is well conserved among eukaryotic cells.

The structure of the integrated T-DNA is similar regardless of the host genome wherein the integration took place. This indicates that the same molecular mechanism of T-DNA integration is used by different eukaryotic species. Recently the proteins that mediate non-homologous T-DNA integration have been identified using the yeast Saccharomyces cerevisiae as a model. These included the yeast Ku70-Ku80 heterodimer, DNA ligase IV and the Mrell, Xrs2, Rad50 complex. These proteins are all known to be involved in double strand break (DSB) repair by non-homologous end-joining. As these proteins are conserved in other eukaryotes including animals and plants, this suggests that the same mechanism of non-homologous T-DNA integration is used by all species studied so far. This demonstrates that Agrobacterium delivery of T-DNA offers an feasible system to study DNA trafficking and DSB repair in eukaryotic cells.

Sample Preparation

At various intervals after the nucleic acid is introduced into the cell, the cell is treated in preparation for in situ hybridization. The appropriate treatment will depend on the type of sample to be examined, as known in the art. During the treatment, the sample will be subject to fixation and, if required, permeabilization.

In one aspect of the invention, the sample is deposited onto a solid support. The particular techniques appropriate for depositing the sample depends on the type of sample. Such techniques include, for example, sectioning of tissue as well as smearing or cytocentrifugation of cell suspensions.

The types of solid supports are known in the art. Supports which may be utilized include, but are not limited to, microporous beads or sponges, glass, Scotch tape (3M), nylon, Gene Screen Plus (New England Nuclear), magnetic particles and nitrocellulose. Most preferably glass microscope slides are used. The use of these supports and the procedures for depositing specimens thereon are known in the art. The choice of support material will depend upon need for the procedure used to visualize or analyze cells and the quantitation procedure used. Some filter materials are not uniformly thick and, thus, shrinking and swelling during in situ hybridization procedures is not uniform. In addition, some supports which autofluoresce will interfere with the determination of low level fluorescence. Glass microscope slides are most preferable as a solid support since they have high signal-to-noise ratios and can be treated to better retain tissue.

Prior to hybridization, the sample is suitably treated with various chemicals to facilitate the subsequent reactions. The actual pretreatment will depend on the type of sample to be analysed and on whether DNA or RNA sequences are to be detected. For monitoring RNA, the sample may need to be treated as soon as possible after the RNA is introduced into the cell. It may be advantageous to treat the sample with DNase to minimise the background noise when the target sequence is RNA. By fixing the cells in the sample, the morphological integrity of the cellular matrix and of the nucleic acids within the cell is preserved.

Fixing may be by means of chemical fixation or freezing. When freezing is used for preservation of a sample, the sample may be frozen in liquid nitrogen and stored at −80° C. Prior to the analysis of the nucleic acid, the frozen sample is cut into thin sections and transferred to e.g. pre-treated slides. This can e.g. be carried out at a temperature of −20° C. in a cryostat. The tissue sections may suitably be stored at −80° C. until use.

In preparation for hybridization, the biological sample may be treated with a fixative, including a precipitating fixative such as acetone. Alternatively, the biological sample is incubated for a short period in a solution of buffered formaldehyde. The biological sample can also be transferred to a fixative such as buffered formaldehyde for 12 to 24 hours. Following fixation, the biological sample may be embedded in paraffin forming a block from which thin sections can be cut.

Fixatives are compounds that kill a cell but preserve its morphology and/or nucleic acids for an extended period of time. They act either by creating covalent linkages between cellular molecules or by precipitating certain intracellular molecules. Cross-linking fixatives include formaldehyde, glutaraldehyde, paraformaldehyde, ethyldimethyl-aminopropyl-carbodiimide, and dimethylsilserimidate. Precipitants include ethanol, acetic acid, methanol, acetone, and combinations thereof. It is further preferred that glacial acetic acid be included as a fixative when the cells are to be monitored by flow cytometry. If a cross-linking fixative is used, paraformaldehyde (0.1% v/v to 4% v/v is preferred, 0.5% v/v to 1% v/v is especially preferred; 2 hours to 20 hours preferred). Formaldehyde and gluteraldehyde are among the other possibilities. Fixatives are used at concentrations which do not destroy the ability of the cell's nucleic acids to hybridize to the probe. Fixatives and hybridization of fixed cells, in general, are discussed in WO 90/02173 and WO 90/02204. See also U.S. Pat. No. 5,719,023, U.S. Pat. No. 5,888,733 and U.S. Pat. No. 5,521,061 for general discussions of in vitro hybridization.

Prior to hybridization, the biological sample may be de-waxed and rehydrated using standard procedures.

For all sample preparation, the nucleic acids are fixed in morphological relationship with cellular structure allowing hybridization to be carried out in situ. The nucleic acids are not extracted from the cellular material and hybridization is not carried out in solution.

If RNA sequences are the target for hybridization, degradation by ribonucleases during the prehybridization steps should be avoided. All equipment and solutions used for pretreatment as well as for hybridization should be appropriately treated to remove nucleases. Such inactivation techniques are well known in the literature and may be performed according to standard procedures.

In preparing a biological sample for in situ hybridization, it may be necessary to treat the sample so as to permeabilize the material and preserve the morphology.

Permeabilization may be necessary in order to ensure sufficient accessibility of the probe to the target nucleic acid sequences. The type of treatment will depend on several factors, for instance on the fixative used, the extent of fixation, the type and size of sample used and the length of the probe. The treatment may involve exposure to protease such as proteinase K, pronase or pepsin, diluted acids, detergents or alcohols or a heat treatment.

For biological samples such as plant where the cells have cell walls, it may be necessary to remove the cell wall to allow the probe access to the target nucleic acid. The cell wall may be removed by digestion with a cell wall-digesting enzyme such as cellulase. In one embodiment, the cell wall is removed after the cells have been fixed, but before permeabilization.

Permeabilizing agents include, but are not limited to, detergents such as Brij 35, Brij 58, sodium dodecyl sulfate, CHAPS, and TRITON X-100. Depending on the location of the target nucleic acid, the permeabilizing agent is chosen to facilitate probe entry through the cell membranes, preferably the plasma membrane. For instance, 0.05% Brij 35 or 0.1% TRITON X-100 will permit probe entry through the plasma membrane but not the nuclear membrane. Alternatively, sodium deoxycholate will allow probes to traverse the nuclear membrane. Thus, in order to restrict hybridization to the cytoplasmic structures, nuclear membrane permeabilizing agents are avoided. Such selective subcellular localization may improve the specificity and sensitivity of detection by minimizing probe hybridization to complementary nuclear sequences when the target sequence is located in the cytoplasm.

In situ Hybridization

In situ hybridization may be performed using any of the methods known in the art (see Jong et al 1999. High resolution FISH in plants—techniques and applications. Trends in Plant Science, 4, 258-263 and Nath J and Johnson KL 2000. A review of fluorescence in situ hybridization (FISH): Current status and future prospects. Biotech Histochem 75: 54-78). The basic steps involve hybridization of a probe to the exogenous nucleic acid, washing the sample to remove non-specific binding, and visualizing the bound probe.

A probe is defined as genetic material DNA, RNA, or oligonucleotides or polynucleotides comprised of DNA or RNA. The DNA or RNA may be composed of the bases adenosine, uridine, thymidine, guanine, cytosine, or any natural or artificial chemical derivatives thereof. The probe is capable of binding to a complementary or mirror image target nucleic acid through one or more types of chemical bonds, usually through hydrogen bond formation. The extent of binding is referred to as the amount of mismatch allowed in the binding or hybridization process; the extent of binding of the probe to the target sequences also relates to the degree of complementarity to the target sequences. The size of the probe is adjusted to be of such size that it forms stable hybrids at the desired level of mismatch; typically, to detect a single base mismatch requires a probe of approximately 12-50 bases. Larger probes (from 50 bases up to tens of thousands of bases) are more often used when the level of mismatch is measured in terms of overall percentage of similarity of the probe to the target cellular genetic sequence. The size of the probe may also be varied to allow or prevent the probe from entering or binding to various regions of the genetic material or of the cell. Similarly, the type of the probe (for example, using RNA versus DNA) may accomplish these objectives. The size of the probe also affects the rate of probe diffusion, probability of finding a cellular target match, etc.

The length of a probe affects its diffusion rate, the rate of hybrid formation, and the stability of hybrids. As a general guide, to detect target RNA, small probes (50-150 bases) may allow the most sensitive, rapid and stable signal. A mixture of short probes (50-150 bases) are prepared which span the entire length of the target sequence. For example, if the target sequence were 1000 bases long, about 10-20 “different” probes of 50-100 bases would be used in the hybrid solution to completely cover all regions of the target sequence. To detect target DNA, smaller probes (15-50 bases) may be utilized.

The concentration of the probe affects several parameters of the in situ hybridization reaction. High concentrations are used to increase diffusion, to reduce the time of the hybridization reaction, and to saturate the available cellular sequences. In an embodiment, probe concentrations of 1-10 μg/ml or 2.5 μg/ml are used.

Nucleic acid probes can be prepared by a variety of methods known in the art. The probe may be constructed or obtained by one of a number of standard methods. Many probes, such as various satellite DNA sequences are commercially available in single-stranded or double-stranded form. Other probes can be obtained either directly from viruses, plasmids and cosmids or other vectors carrying specific sequences, or, if desired, by restriction digest of the source of the probe DNA, such as a vector, followed by electrophoretic isolation of specific restriction digestion fragments. Probes obtained in this manner are typically in double-stranded form, but may, if required, be subcloned in single-stranded vectors, such as an M13 phage vector.

Purified double-stranded sequences of DNA (dsDNA) can be labeled intact by the process of nick translation or random primer extension. The ability of double-stranded probes to hybridize to nucleic acids immobilized within cells is compromised by the ability of the complementary strands to hybridize to each other in solution prior to hybridization with the cellular nucleic acids. Single-stranded DNA (ssDNA) probes do not suffer this limitation and may be produced by the synthesis of oligonucleotides, by the use of the single-stranded phage M13 or plasmid derivatives of this phage, or by reverse transcription of a purified RNA template. The use of single-stranded RNA (ssRNA) probes in hybridization reactions potentially provides greater signal-to-noise ratios than the use of either double or single-stranded DNA probes. Regardless of whether a dsDNA, a ssDNA, or a ssRNA probe is used in the hybridization reaction, there must be some means of detecting hybrid formation. The means of detecting hybrid formation utilizes a probe “labeled” with some type of detectable label.

For a discussion of probes, see Handbook of Fluorescent Probes and Research Products, Ninth Edition by Richard P. Haugland (2002) Molecular Probes.

The probe is labeled with a reporter or ligand or moiety which allows detection of the targeted sequence in situ. The probes may be detectably labeled prior to addition to the hybridization solution. Alternatively, a detectable label may be selected which binds to the reaction product. Probes may be labeled with any detectable group for use in practicing the invention. Such detectable group can be any material having a detectable physical or chemical property. Such detectable labels have been well-developed in the field of immunoassays and in general most any label useful in such methods can be applied to the present invention. Particularly useful are enzymatically active groups, such as enzymes (see Clin. Chem., 22:1243 (1976)), enzyme substrates (see British Pat. Spec. 1,548,741), coenzymes (see U.S. Pat. Nos. 4,230,797 and 4,238,565) and enzyme inhibitors (see U.S. Pat. No. 4,134,792); fluorescers (see Clin. Chem., 25:353 (1979); chromophores; luminescers such as chemiluminescers and bioluminescers (see Clin. Chem., 25:512 (1979)); specifically bindable ligands; proximal interacting pairs; and radioisotopes such as ³H, ³⁵S, ³²P, ¹²⁵I and ¹⁴C. For autoradiographic detection, the reporter is a radiolabel, such as ³²P-labeled probe formed, for example by nick translation or polymerase chain reaction in the presence of labeled nucleotides.

For fluorescence detection, the probe may be labeled with one of a selection of fluorescence groups, such as FITC, BODIPY, Texas Red, or Cascade Blue which is excitable in a specific wavelength, such as 490, 540, and 361 nm. The groups are derivatized to 3′ or 5′ probe ends or by incorporation or reaction at internal positions, according to standard methods.

Alternatively, the probes may be labeled with a ligand-type reporter such as biotin, digoxigenin, or bromodeoxyuridine or other modified bases including fluorescein-11-dUTP. The probe reporter groups are detected, in situ, by reaction of the hybridized probe with a secondary reporter molecule which (a) binds specifically and with high affinity to the probe ligands, and (b) contains a detectable reporter. The binding moiety of the secondary molecule may be avidin or streptavidin, for binding to biotinylated nucleotides, anti-digoxigenin antibody, for binding to digoxigenin-labeled nucleotides, and anti-BrdUrd antibody for binding to BrdUrd-labeled probe.

The detectable reporter in the secondary molecule is typically a fluorescence label, but may also be a radiolabel, for autoradiographic detection, an antibody, an enzyme, for colorimeteric or chemiluminescence detection in the presence of a suitable substrate, or colloidal gold for use in electron microscopic visualization.

Nucleic acid hybridization is a process well known in the art where two or more mirror images or opposite strands of DNA, RNA, oligonucleotides, polynucleotides, or any combination thereof recognize one another and bind together through the formation of some form of either spontaneous or induced chemical bond, usually a hydrogen bond. The degree of binding can be controlled based on the types of nucleic acids coming together, and the extent of “correct” binding as defined by normal nucleic acids coming together, and the extent of “correct” binding as defined by normal chemical rules of bonding and pairing.

Hybridization of the probe to the target introduced nucleic acid can be performed using the fixed and permeabilized preparations prepared as described above. If double-stranded target such as chromosomal or DNA sequences are to be detected, a treatment to separate the two strands may be necessary. This separation of the strands can be achieved by heating the sample in the presence of the hybridization mixture to a temperature sufficiently high and for a time period sufficiently long to dissociate the strands. Typically, heating at a temperature of 90° C. to 95° C. for a period of 5 to 15 minutes is suitable.

The hybridization buffer may contain a hybrid destabilizing agent in an amount effective to decrease the melting temperature of hybrids formed between the nucleic acid to be determined and the binding partner so as to increase the ratio between specific binding and non-specific binding (see U.S. Pat. No. 5,888,733). This agent allows the hybridization to take place at a lower temperature than without the agent. In traditional nucleic acid hybridization, such agent is called a denaturing agent. Hybridization and denaturing may be carried out simultaneously using a suitable amount a hybrid destabilizing agent in combination with a suitable temperature for the treatment. Examples of hybrid destabilizing agents are formamide, ethylene glycol and glycerol and these agents may preferably be used in a concentration above 10% and less than 70%. The concentration of formamide may more preferably be from 20% to 60%, most preferably from 30% to 50%. The concentration of ethylene glycol may more preferably be from 30% to 65%, most preferably 65%. The concentration of glycerol may more preferably be from 45% to 60%, most preferably 50%.

It is often advantageous to include macromolecules or polymers such as dextran sulphate, polyvinylpyrrolidone and/or ficoll. In the presence of such macromolecules or polymers, the effective concentration of the probe at the target is assumed to be increased. Dextran sulphate may be added in a concentration of up to 15%. Concentrations of dextran sulphate of from 2.5% to 12.5% may be advantageous.

Other important hybridization parameters are temperature, concentration of the probe and hybridization time. A skilled person will readily recognize that optimal conditions for various starting materials will have to be determined for each of the above-mentioned parameters.

Following hybridization, the prepared biological sample is washed to remove any unbound and any non-specifically bound probes. During the post-hybridization step, appropriate stringency conditions should be used in order to remove any non-specifically bound probe. Stringency refers to the degree to which reaction conditions favour the dissociation of the formed hybrids and may be enhanced, for instance by increasing the washing temperature and incubation time. Salt concentration is often used as an additional factor for regulating the stringency. Examples of useful buffer systems are Tris-Buffered-Saline (TBS), standard citrate buffer (SSC) or phosphate buffers. A convenient TBS buffer is 0.05M Tris/HCl, 0.15M NaCl, pH 7.6. The SSC buffer comprises 0.15M sodium chloride and 0.015M trisodium citrate, pH 7.0. Typically, washing times from 25 to 30 minutes may be suitable. Washing periods of two times 10 minutes or 3 times 5 minutes in a suitable buffer may also be suitable.

Where the preparation is deposited onto slides, the hybridization results may be visualized using well known immunohistochemical staining methods to detect the labelling on the probe. When fluorescent labelled probes are used, the hybrids may be detected using an antibody against the fluorescent label which antibody may be conjugated with an enzyme. The fluorescent label may alternatively be detected directly using a fluorescence microscope, or the results may be automatically analysed on a fluorescent-based image analysis system. The signal may be visualized by confocal microscopy, fluorescence microscopy, or electron microscopy.

When biotin labelled binding partners are used, the hybrids may be detected using an antibody against the biotin label which antibody may be conjugated with an enzyme. If necessary, an enhancement of the signal can be generated using commercially available amplification systems such as the catalyzed signal amplification system for biotinylated probes (DAKO K 1500).

Applications of VOID

VOID is useful for monitoring introduced nucleic acids whenever the nucleic acid is in transit. As examples, a number of specific applications are contemplated below.

Applications of VOID utilize VOID's ability to reveal the number and location of the introduced nucleic acid, as well as its ability to identify the cells that are competent for receiving nucleic acid molecules. In addition, VOID can be used to determine the fate and timing of nucleic acid being delivered into the cells; that is, VOID can be used to identify where the nucleic acid molecules have gone after the nucleic acid has entered the cell and where they are in the cell at any time after cell entry. By the use of VOID, drawbacks associated with current techniques for monitoring nucleic acid delivery and transformation may be avoided.

VOID can be used to help the development of genetically modified products, including gene therapy vectors, gene therapy delivery systems, as well as assess the safety of gene therapy treatments. VOID can also be used to facilitate development of DNA vaccines, DNA vaccine vectors, DNA vaccine delivery systems, and safety assessment of DNA vaccines. In addition, VOID can be used in the development of transgenic or genetic engineered products such as genetically modified food crops.

The use of VOID in genetic modification is significant because for development of safe and effective genetically modified products or gene delivery vectors such as those used in gene therapy, one needs to know the number, location and fate of the nucleic acid delivered into the targeted cells. Because VOID can reveal the number and location of the nucleic acid delivered into the cells and identify the cell types competent for receiving the nucleic acid, this facilitates identification and development of DNA delivery systems and formulations, and generate efficient nucleic acid delivery where the desired number of nucleic acid molecules are delivered into the desired target cells. Thus use of VOID can considerably speed up the development of genetically modified products including gene therapy products and DNA vaccines.

The use of VOID in the development of transgenic or genetic engineered products is also significant because marker or reporter genes are no longer necessary to monitor nucleic acid delivery and transformation. As a result, transgenic products may be produced that are free of marker or reporter genes which may be of concern for the environment and human health.

VOID can also be used to determine the cell types that can efficiently receive the nucleic acid intended for transformation, thus identifying the best cells and tissues or organs for nucleic acid uptake. As illustrated below with the cellular protein VirD2-Interacting protein (VDI; SEQ ID NO:1), VOID is also useful for identifying molecular markers associated with a cell's competence to receive DNA molecules.

I. Assessing Transformation Status and Efficiency

In one embodiment, VOID is used to monitor T-DNA introduced into plant cells by A. tumefaciens.

Agrobacterium tumefaciens is a natural genetic engineer that transforms plants with its own genes for its own benefits (P. J. Christie. Mol. Microbiol, 40:294 (2001); S. B. Gelvin. Annu. Rev. Plant Physiol. Plant Mol. Biol. 51:223 (2000); J. Zhu et al. J. Bacteriol. 182:3885 (2000); J. Zupan et al. Plant J. 23:11 (2000)). The bacterium transfers a specific segment (T-DNA) of its tumor-inducing (Ti) plasmid into plant cells, where the T-DNA becomes integrated into the plant genome. This system has been used as the workhorse to introduce various genes into many different plant species.

Recently, it has been demonstrated that Agrobacterium can also transfer T-DNA into yeast (Bundock et al. 1995. EMBO J. 14:3206-14; Piers et al. 1996. Proc Natl Acad Sci USA 93:1613-8), fungal (de Groot M J et al. 1998. Nat Biotechnol. 16:839-42) and human cells (Kunik et al. 2001. Proc Natl Acad Sci USA. 98:1871-1876). Major bacterial genes involved in the gene transfer process have been identified and characterized (Christie, P. J., 2001. Mol. Microbiol., 40(2), 294-305; Gelvin, S. B., 2000. Annu. Rev. Plant Physiol. Plant Mol. Biol., 51, 223-256; Zhu et al. 2000. J. Bacteriol., 182, 3885-3895; Zupan et al. 2000. Plant J., 23, 11-28).

A. tumefaciens genome has been sequenced (Wood, D. W., et al 2001. Science, 294, 2317-2323; Goodner, B., et al 2001. Science, 294, 2323-2328). The Ti plasmid harbored virulence (vir) genes are directly responsible for the processing and transfer of T-DNA; all of them are induced by plant signal molecules, such as acetosyringone (AS). VirD2 generates a linear single-stranded (ss) DNA molecule (the T-strand) by nicking the border repeats that delineate the T-DNA (Stachel, S. E., Nester, E. W., 1986. EMBO J., 5, 1445-1454; Yanofsky G, et al. Cell. Nov. 7, 1986;47(3):471-7). The T-strand is transferred into plant nuclei presumably in the form of nucleoprotein complex (T-complex) (Ziemienowicz et al. 2001. The Plant Cell, 13, 369-384) consisting of one VirD2 molecule, one T-strand and many VirE2 molecules. The virB gene products and VirD4 form a membrane structure responsible for transferring the T-DNA (Fullner et al. 1996. 273, 1107-1109) in a process mechanistically analogous to conjugation (Christie, P. J., 2001. Mol. Microbiol., 40(2), 294-305) and promiscuous enough to be applicable to exceptionally diverse recipients like plant, yeast, fungal and human cells.

Plant proteins that interact with VirD2 and VirE2 have been identified (Ballas, N., Citovsky, V., 1997. Nuclear localization signal binding protein from Arabidopsis mediates nuclear import of Agrobacterium VirD2 protein. Proc. Natl. Acad. Sci. USA, 94, 10723-10728; Deng et al. 1998. Agrobacterium VirD2 protein interacts with plant host cyclophilins. Proc. Natl. Acad. Sci. USA, 95, 7040-7045; Tzfira et al. 2001. VIP1, an Arabidopsis protein that interacts with Agrobacterium VirE2, is involved in VirE2 nuclear import and Agrobacterium infectivity, EMBO J., 20, 3596-3607); some plant proteins have been implicated in the nuclear import of T-complex or T-DNA integration (Gelvin, S. B. 2000. Annu. Rev. Plant Physiol. Plant Mol. Biol., 51, 223-256). However, individual T-DNA molecules have never been visualized inside eukaryotic cells during the T-DNA passage in vivo.

It is still unknown how many T-DNA molecules can be delivered into a single eukaryotic cell and how many of them can be transferred from cytoplasm into nucleus. The mode of T-DNA passage into and through eukaryotic cytoplasm remains elusive. It is not well established why some eukaryotes including certain monocotyledonous plants are recalcitrant to Agrobacterium-mediated transformation, while many receptive eukaryotes like dicotyledonous plants can be efficiently transformed, with a variable number of integrated T-DNA copies. To help address these questions, VOID was used to monitor T-DNA molecules trafficking inside plant cells. This procedure may be used not only to dissect the T-DNA trafficking pathway(s) inside eukaryotic cells, but also to monitor the T-DNA transfer and passage inside eukaryotic cells.

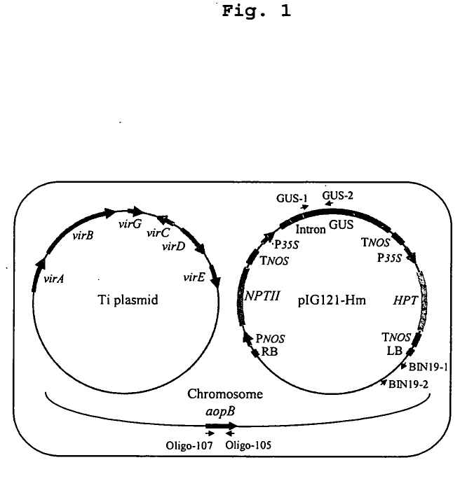

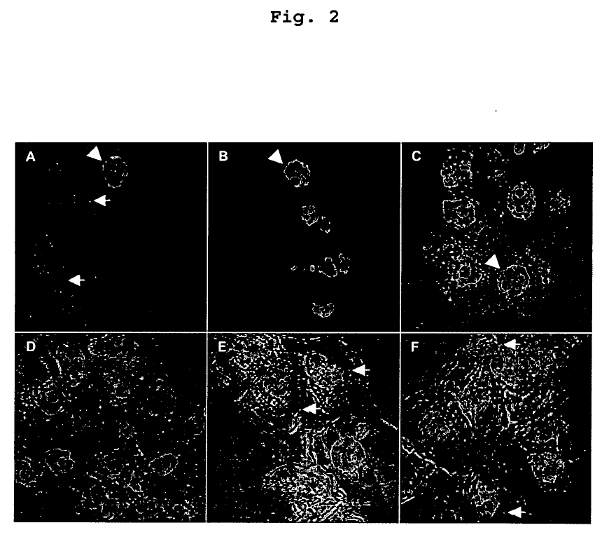

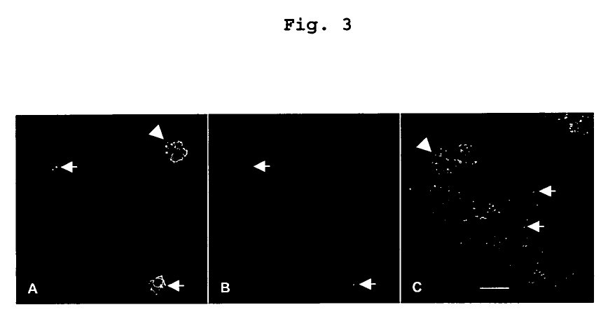

Part of a T-DNA fragment was labeled with digoxigenin-11-dUTP (DIG); the T-DNA probe was allowed to permeate the plant cells that had been cocultivated with A. tumefaciens cells harboring a vector plasmid (pIG121-Hm) containing the T-DNA (FIG. 1). The T-DNA molecules delivered into the plant cells were allowed to hybridize with the probe DNA labeled with DIG, which could be bound to anti-DIG antibody conjugated with rhodamine which gave red fluorescence when excited by 543 nm light. As shown in FIG. 2A, many red dots could indeed be detected under confocal microscope when the tobacco BY2 cells were allowed to cocultivate with LBA4404(pIG121-Hm). The nuclei were counterstained with PicoGreen, which could give green fluorescence, in order to gauge the subcellular locations of T-DNA molecules.

To verify that the red dots indeed corresponded to the T-DNA inside plant cells rather than T-DNA from contaminating A. tumefaciens, the following experiments have been conducted. First, the bacteria were extensively washed away after the cocultivation; indeed very few bacteria could be found after the cocultivation and then the VOID procedure. Second, it was determined if the red dots were specifically correlated with the bacterial ability to deliver the T-DNA. The same plasmid pIG121-Hm was introduced into a virb mutant MX243 (Stachel & Nester, E. W., 1986. EMBO J., 5, 1445-1454; P. J. Christie. Mol. Microbiol. 2001. 40:294), which is unable to deliver the T-DNA, and a virD2 mutant WR1715 (Stachel & Nester, E. W., 1986. EMBO J., 5, 1445-1454; Shurvinton et al. 1992. Proc. Natl. Acad. Sci. USA, 89, 11837-11841; Zupan et al. 2000. Plant J., 23, 11-28), which is unable to produce the T-DNA. When the cocultivation and VOID were conducted with these mutants, no T-DNA signal was detected inside plant cells (FIG. 2B&C). This indicated that the signal was due to the hybridization of T-DNA with the specific probe.

Finally, it was determined if the signal was due to the untransferred T-DNA still residing inside A. tumefaciens cells. The A. tumefaciens chromosomal gene aopB (Jia Y. H., L. P. Li, Q. M. Hou and S. Q. Pan. 2002. An Agrobacterium gene involved in tumorigenesis encodes an outer membrane protein exposed on the bacterial cell surface. Gene. 284:113-124) was labelled and the same VOID procedure was carried out. As shown in FIG. 2D, no signal was detectable in the samples when the aopb probe was used for hybridization. This indicated that the VOID procedure could not detect any DNA residing inside the bacterial cells, presumably because of insufficient permeabilization of the bacterial membranes. These confirmed that the VOID procedure could detect the T-DNA delivered into plant cells.

II. Numbers and Locations of DNA Molecules Delivered into Cells

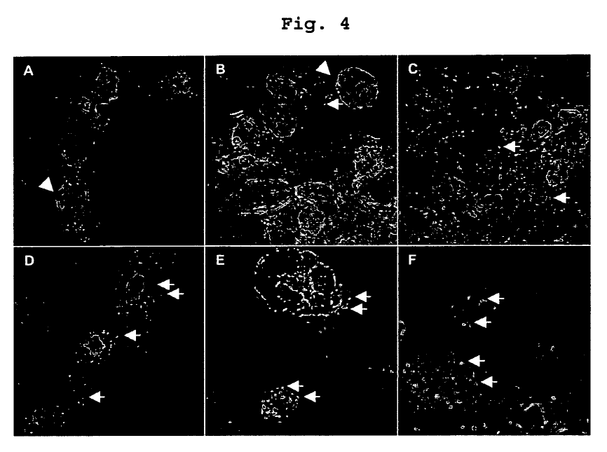

Subsequently, it was important to know if the VOID procedure could reveal the location and number of individual T-DNA molecules. To do this, an Arabidopsis thaliana cDNA (corresponding to F16N3.18 of the genome sequence) clone was chosen that could hybridize to the BY2 genomic DNA as demonstrated by Southern analysis. When this cDNA fragment was used to probe BY2 cells, VOID consistently revealed 1 or 2 red dots in each single cell nucleus (FIG. 3A&B). No hybridization signal was detected in the cytoplasm of BY2 cells, suggesting that VOID could detect DNA and not RNA molecules as RNase A was added. Conceivably, 2 red dots could been seen if the confocal section in focus contained two nuclear DNA molecules that could specifically hybridize with the cDNA fragment; 1 red dot would be seen if only one was in focus. This indicated that VOID could reveal the locations and numbers of individual T-DNA molecules present throughout the BY2 cells.

When a virE2 mutant MX358 (Stachel & Nester. 1986. EMBO J., 5, 1445-1454; Winans et al. 1987. Nucleic Acid Research 15: 825-836; Zupan et al. 2000. Plant J., 23, 11-28) was used to conduct the cocultivation and VOID procedure, very few T-DNA molecules were found inside the BY2 cells (FIG. 2E). This suggests that mutation at virE2 may severely attenuate the bacterial ability to deliver the T-DNA into plant cells, which is consistent with the recent evidence that the VirE2 protein can form a membrane channel to facilitate ssDNA transport (Dumas et al. 2001. Proc. Natl. Acad. Sci. USA, 98, 485-490). It may also indicate that T-DNA molecules were quickly degraded inside plant cells in the absence of the VirE2 protein, which is known to bind to T-DNA and presumably can protect T-DNA from degradation (Zhu et al. 2000. J. Bacteriol., 182, 3885-3895; Zupan et al. 2000. Plant J., 23, 11-28).

To determine whether DNA segments outside the T-DNA could also be transferred into the plant cells, a fragment of pIG121-Hm vector backbone outside the left border (FIG. 1) was used to probe the BY2 cells cocultivated with LBA4404(pIG121-Hm). As shown in FIG. 2F, the vector backbone fragment outside the T-DNA were detected in the plant cells. However, the numbers of such molecules appeared to be about 18 times lower as compared to the T-DNA molecules. This suggests that the binary vector backbone can be transferred into the plant cells along with the T-DNA, although the frequency of such an event is lower than that for the T-DNA alone. In fact, previous experiments demonstrated that A. tumefaciens could transfer DNA sequences outside the T-region and even plasmids into plant cells (Gardner and Knauf. Science, Vol. 231, No. 4739. Feb. 14, 1986, pp. 725-727; Buchanan-Wollaston et al. 1987. Nature 328: 172-175). These further demonstrated that the VOID procedure could reliably reveal data consistent with the previous observations.

The numbers of T-DNA molecules inside BY2 cells were counted after reconstitution of individual plant cells from sequential 1-μm-laser-sections of confocal microscopy. Roughly half of the BY2 cells did not contain any T-DNA molecules, suggesting that the competency of BY2 cells was important for BY2 cells to receive the T-DNA delivered by A. tumefaciens. The numbers of T-DNA molecules inside the BY2 cells that had received T-DNA also varied greatly. Some received hundreds of T-DNA molecules (FIG. 2A); some had only a few (FIG. 3C). In a typical cocultivation experiment, the average numbers of T-DNA molecules inside the cytoplasm of a single BY2 cell that received T-DNA were around 63.

III. Time-Course of DNA Delivery into Cells

BY2 cells were cocultivated with LBA4404(pIG121-Hm) for different time intervals. As shown in FIG. 4B, the T-DNA was barely detectable in the plant cytoplasm at 2 h of cocultivation, which is consistent with the previous observations that T-DNA could be detected at 2 h after infection of plants with A. tumefaciens (Virts & Gelvin. 1985. J. Bacteriol., 162, 1030-8; Narasimhulu et al. 1996. Plant Cell 8: 873-886). Most of the T-DNA molecules entered the plant cytoplasm in 5 h (FIG. 4C). At 1 day of cocultivation, all the T-DNA molecules still remained in the cytoplasm of the BY2 cells (FIG. 4D). At 2 days of cocultivation, all the T-DNA molecules were inside the plant nuclei (FIG. 4E). This is consistent with the β-glucuronidase (GUS) staining experiments, which could indicate the expression of T-DNA. It was found that the GUS activity was not detectable until 2 days of cocultivation (data not shown). These suggest that the T-DNA could enter plant cells very fast (about 5 h), but it took a longer period (about 2 d) for T-DNA to enter plant nuclei. The T-DNA harbored genes were quickly expressed upon entry of T-DNA into the nuclei.

IV. Eliminating need for Selection/Marker to Identify Transformant

Widespread use and the subsequent spreading of specific marker genes in genetically modified products has raised concerns about the safety and environmental effects of these products. Currently genetically modified cells are selected on the basis of expression of a functional product of the DNA delivered; the functional product may be the desired product, or more commonly, the product of a gene that is introduced into the cell along with the gene encoding the desired product. These latter products are commonly referred to as selection markers or reporters.

Selection markers are those genes which, upon expression, produces a protein capable of facilitating the isolation of cells expressing the marker. Examples of markers include neomycin, hypoxanthine phosphribosyl transferase, puromycin, dihydrooratase, glutamine synthetase, histidine D, carbamyl phosphate synthase, dihydrofolate reductase, multidrug resistance I, aspartate transcarbamylase, xanthine-guanine phosphoribosyl transferase, or adenosine deaminase. In plants, markers are used that confer on the transformed plant cells resistance to a biocide or an antibiotic, such as kanamycin, G 418, bleomycin, hygromycin, or chloramphenicol, etc.

Reporter genes encode a functional product such that when the gene is expressed, the product is detectable by means of a suitable assay. Common reporter genes include genes encoding luciferase, beta-galactosidase, chloramphenicol acetyl transferase, secreted alkaline phosphatase or Green Fluorescent Protein (GFP) gene.

Since the function of the selection marker or reporter is to permit identification and selection of the transformed cell, the marker or reporter becomes unnecessary after the transformant is identified. Thus ideally the transformed cell would contain only the desired nucleic acid, with as little as possible of non-essential material, such as marker genes and remnants of the DNA used for cloning.

In one embodiment, VOID is used to identify cells that contain the T-DNA introduced into plant cells by A. tumefaciens. Identification of transformed cells is determined by monitoring entry of the T-DNA into the nucleus. As shown in FIG. 4C, most of the T-DNA molecules entered the cytoplasm in 5 h. However, none of them entered the nucleus even at 1 day of cocultivation (FIG. 4D). Once they entered the nucleus in 2 days, no T-DNA molecule could be found in the cytoplasm (FIG. 4E). These results indicate that the T-DNA molecules appeared to have moved into the nucleus together in one wave. The data also suggest that the T-DNA trafficking inside plant cells is not a simple diffusion process. If it was a diffusion process, some T-DNA molecules would have arrived inside the nuclei at 1 day of cocultivation, since many T-DNA molecules were already present throughout the cytoplasm at 5 h of co-cultivation. In addition, some of them would have remained inside the cytoplasm while others entered the nucleus.

Previous experiments with an in vitro nuclear import system demonstrated that T-complex could be translocated from plant cytoplasm into nucleus very quickly (within 20 min) (Ziemienowicz et al. 2001. The Plant Cell, 13, 369-384). The in vivo data indicated that T-DNA trafficking inside plant cytoplasm was very slow. It appeared that the T-DNA molecules inside cytoplasm were somehow unavailable for or prohibited from the quick nuclear import process. Consistent with this, T-DNA trafficking inside plant cytoplasm appeared to occur in a coordinated manner, as only one wave of T-DNA trafficking was apparent during the 3-day cocultivation (FIG. 4).

Thus by using VOID, the cycle of T-DNA import into the nucleus is observed, and cells which have undergone T-DNA import can be identified without having to use a selection or marker product.

V. Assessing Risk Associated with Transformation or Nucleic Acid Delivery

It is important to know how many DNA molecules have been delivered into the cells and where the molecules have gone. This is because an excess number of DNA molecules delivered into the cells could potentially lead to unwanted integration of some of the DNA molecules into vital sites of the genome, which could lead to serious health problems to the host. Since VOID can be used to determine the number and location of the nucleic acid delivered into the cells, VOID can be used to calculate the risk associated with a genetically modified product transformed using a particular gene delivery process.

By monitoring the fate of the exogenous nucleic acid in the cell, one can assess the relative safety not only of a particular gene delivery technique, but also the risk associated with using a particular nucleic acid delivery vehicle. For example, the uptake, number, location, and movement of a desired gene may be monitored when the gene is delivered as part of a naked plasmid, as part of a liposomal complex, as part of a viral delivery system such as retroviral vectors, adeno associated viral vectors, herpes simplex viral vectors, and bacteriophage vectors; as part of a nucleic acid associated with cationic peptides, or as part of an Agrobacterium tumefaciens vector. Depending on the context in which the desired gene is delivered, the desired gene may be stable or unstable in the cell; the gene may also be capable of replicating at different rates and efficiency. All these factors are important in assessing the desirability of using a particular gene delivery system.

In one embodiment, VOID is used to assess the risk associated with A. tumefaciens-mediated transformation of plant cells. The efficiency of T-DNA molecules that moved from cytoplasm into nucleus was assessed by determining the ratio of the numbers of T-DNA molecules inside cytoplasm to those inside nucleus. In a typical cocultivation, it was found that 1 in 6 T-DNA molecules moved from cytoplasm to nucleus (FIG. 4D&E). This demonstrated that not every T-DNA molecule delivered into plant cytoplasm could make all the way into the nucleus, suggesting that the transformation process involved a shot-gun approach. Since the VOID procedure could reveal the number and location of T-DNA molecules trafficking inside plant cells, VOID is useful for calculating risks associated with certain transformation or DNA delivery processes.

Since 1 out of 6 T-DNA molecules moved from the cytoplasm into the nucleus, 6 T-DNA molecules would be the minimal number of DNA molecules that need to be delivered into the cytoplasm per cell in order to generate a transgenic plant containing one copy of the transgene in the plant genome. If 60 T-DNA molecules were delivered into cytoplasm per cell, the risk of generating a transgenic plant containing multiple copies of the transgene would increase 10 fold. Similarly, the risk of generating a transgenic plant containing the transgene inserted at an undesired site would also increase 10 fold. If there were 600 T-DNA molecules delivered into cytoplasm per cell, the risk of generating an undesired transgenic product would increase 100 fold, although the transformation efficiency would also increase. One must compromise between transformation efficiency and the risk of an undesired outcome. The VOID procedure can facilitate the determination of an appropriate compromise in the early stage of product development.

The risk associated with a particular gene delivery vehicle such as a gene therapy vector or a DNA vaccine vector may be assessed in a similar fashion. For example, a gene therapy vector may be used to treat a test subject. The number and location of the nucleic acid molecules inside the treated cells may be determined as described above, at various time intervals after the treatment. The ratio of the nucleic acid molecules present in the cytoplasm to those in the nucleus can be calculated. The risk associated with the treatment can be then assessed as described above.

VI. Controlling the Copy Number of Nucleic Acid Molecules Delivered

During the development of a transgenic product or gene delivery vehicle, it is important to control the copy number of nucleic acid molecules delivered into target cells. In some circumstances, a high copy number of the nucleic acid molecules may be desired in order to have a high expression level of products encoded by the nucleic acid. In other circumstances, only one copy of the nucleic acid molecule may be desired per target cell. Since VOID can reveal the number and location of nucleic acid molecules delivered during the early stage of development, VOID may be used to control the copy number of the nucleic acid molecules. This may be achieved by manipulating the parameters associated with the nucleic acid delivery, for instances, methods of nucleic acid delivery, timing, length of time and conditions of the delivery, and conditions (and types) of target cells.

In one embodiment, the number of T-DNA molecules delivered by Agrobacterium could be controlled by the length of time for co-cultivation. BY2 cells were cocultivated with LBA4404(pIG121-Hm) for different time intervals. As shown in FIG. 4B, the T-DNA was barely detectable in the plant cytoplasm at 2 h of cocultivation, which is consistent with previous observations that T-DNA could be detected at 2 h after infection of plants with A. tumefaciens (Virts & Gelvin. 1985. J. Bacteriol., 162, 1030-8; Narasimhulu et al. 1996. Plant Cell 8: 873-886). Most of the T-DNA molecules entered the plant cytoplasm in 5 h (FIG. 4C). At 1 day of cocultivation, all the T-DNA molecules still remained in the cytoplasm of the BY2 cells (FIG. 4D). At 2 days of cocultivation, all the T-DNA molecules were inside the plant nuclei (FIG. 4E). This demonstrates that the number of T-DNA molecules delivered into cytoplasm can be controlled by choosing the appropriate length of time for co-cultivation. Thus an appropriate copy number of the transgene in the target cell can be achieved.

In another embodiment, the number of T-DNA molecules delivered by Agrobacterium could be controlled by the use of different bacterial strains to deliver the T-DNA molecules. When a virE2 mutant MX358 (Stachel & Nester. 1986. EMBO J., 5, 1445-1454; Winans et al. 1987. Nucleic Acid Research 15: 825-836; Zupan et al. 2000. Plant J., 23, 11-28) was used to conduct the cocultivation and VOID procedure, very few T-DNA molecules were found inside the BY2 cells (FIG. 2E). By contrast, when a wild-type Agrobacterium strain was used, some plant cells received hundreds of T-DNA molecules (FIG. 2A). This suggests that mutation at virE2 may severely attenuate the bacterial ability to deliver the T-DNA into plant cells and demonstrates that DNA delivery depends in part on the bacterial strains used.

The finding that virE2 can attenuate T-DNA delivery is consistent with the recent evidence that the VirE2 protein can form a membrane channel to facilitate ssDNA transport (Dumas et al. 2001. Proc. Natl. Acad. Sci. USA, 98, 485-490). It may also indicate that T-DNA molecules were quickly degraded inside plant cells in the absence of the VirE2 protein, since VirE2 is known to bind to T-DNA and presumably can protect T-DNA from degradation (Zhu et al. 2000. J. Bacteriol., 182, 3885-3895; Zupan et al. 2000. Plant J., 23, 11-28).

While some plant cells received hundreds of T-DNA molecules when a wild-type Agrobacterium strain was used (FIG. 2A), some receive only a few T-DNA (FIG. 3C). In a typical cocultivation experiment, the average numbers of T-DNA molecules inside the cytoplasm of a single BY2 cell that received T-DNA were around 63. This demonstrates that DNA delivery depends not only on the bacterial strains, but also on the target cells. In one embodiment, particular strains of Agrobacterium may be selected for a particular target cell population so that an appropriate number of the transgene is delivered into the cells.

VII. Screening for Molecular Markers Associated with Transformation and Identifying VDI and Sec3 Homologs as Molecular Markers

In the context of this invention, molecular markers are proteins which are required for, or assist in, the delivery of introduced DNA into the nucleus; thus molecular markers identify cells which are competent to receive exogenous nucleic acid.

In identifying molecular markers associated with transformation competency, cells into which an exogenous nucleic acid has been introduced are monitored with VOID. The cells are further assayed for co-localization of a cellular protein with the exogenous nucleic acid to determine whether the cellular protein is consistently in close proximity to the exogenous nucleic acid. Co-localization would indicate that the cellular protein is a molecular marker associated with competency to receive exogenous nucleic acid.

In one embodiment, the cellular protein and its location in the cell are identified by immunohistology, using an antibody which binds specifically to the cellular protein. The antibody must be able to bind to the cellular protein (the antigen) in the fixed cell; usually, but not always, this means the antibody must be able to bind to the denatured form of the cellular protein.

In the context of this invention, the cellular protein is any protein which is located in the cytoplasm, the cytosol, the plasma membrane, or the nuclear membrane. Preferably the protein has a surface-exposed or cytoplasm-exposed domain if the protein is located in the plasma membrane. Preferably the protein has a cytoplasm-exposed domain if the protein is located in the nuclear membrane.

In a preferred embodiment, the cellular proteins to be screened as a molecular marker are those which interact with VirD2 or VirE2 (Gelvin. 2000. Annu. Rev. Plant Physiol. Plant Mol. Biol., 51, 223-256). In another embodiment, the cellular protein is VIP1 (Tzfira et al. 2001. EMBO J., 20, 3596-3607), cyclophilins (Deng et al. 1998. Proc. Natl. Acad. Sci. USA, 95, 7040-7045), including specifically cyclophilins having Genbank accession numbers L14844, L14845 and U07276, AtKAPα (Ballas, N., Citovsky, V. 1997. Proc. Natl. Acad. Sci. USA, 94, 10723-10728), and homologs of Sec3, including human, Drosophila, rat, mouse and Caenorhabditis elegans Sec3 (human exocyst component Sec3 Accession number Q9NV70 and homolog accession number NP_{—}060731.1; Drosophila accession number Q9VVG4; rat accession number XP_{—}223340.1 and XP_{—}223339.1; mouse accession number AAH24678.1; Caenorhabditis elegans accession number NP_{—}508530.1). In a another embodiment, the cellular protein is a protein of the Exocyst complex.

Immunohistology is performed according to methods known in the art (see for example U.S. Pat. No. 5,869,274). In one method, a target antigen present in the sample is detected by a double antibody system. Initially the sample is incubated with a primary targeting antibody that is specific for the target antigen. Detection of antigen-antibody complexes containing the primary antibody and formed during the first incubation is accomplished by incubation with a second detecting antibody that binds to a region of the constant domain in the primary antibody; the second antibody is labeled. The result of the second incubation is, in the presence of the target antigen, a complex of antigen and layers of antibodies that contain the label.

In one embodiment, VOID is used to identify molecular markers associated with A. tumefaciens-mediated transformation of plant cells.

BY2 cells that had undergone co-cultivation with A. tumefaciens appear to be clustered in groups which either could, or could not, receive T-DNA. Roughly half of the BY2 cells did not contain any T-DNA molecules, suggesting that the competency of BY2 cells was important for BY2 cells to receive the T-DNA delivered by A. tumefaciens. When the cells received the T-DNA, often all the cells in the same cluster received T-DNA; otherwise, none of the cells in the same cluster received any T-DNA. The BY2 cells were undifferentiated; perhaps, the cell cycle stage may account for the competency to receive T-DNA. VOID could thus facilitate investigations on the plant cell competency to receive T-DNA.

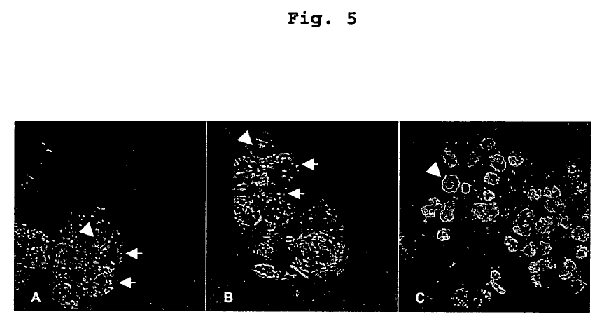

In order to screen for plant proteins that may be important for receiving T-DNA molecules, the plant protein VDI (SEQ ID NO:1) that interacts with A. tumefaciens guide protein VirD2 was investigated as a candidate molecular marker by studying its position inside plant cells during T-DNA delivery. To localize VDI in A. thaliana and tobacco BY2 cells, immunohistology was conducted with anti-VDI as the primary antibody and anti-rabbit IgG-conjugated with Cy3 as the secondary antibody. As shown in FIG. 5, VDI was localized in the cytoplasm of BY2 (FIG. 5A) and A. thaliana cells (FIG. 5B), whereas no signal was detected in the control when preimmune serum instead of anti-VDI was used (FIG. 5C).

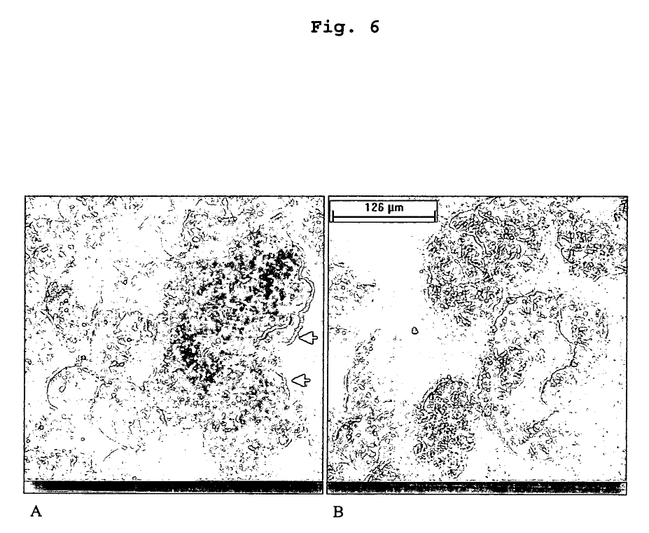

Surprisingly, VDI was not expressed uniformly in all the plant cells. Some cells produced much more VDI than others; many cells did not produce a detectable level of VDI proteins (FIG. 5). The BY2 cells were undifferentiated; it is possible that the cell cycle stage may account for this phenomenon, as the individual cells are undergoing different stages in cell cycling. This uneven expression of VDI in plant cells may explain why only certain cells rather than all BY2 cells could be transformed by A. tumefaciens (FIG. 6). In addition, this might be related to why a high transformation efficiency is normally associated with freshly subcultured BY2 cells. Perhaps only these freshly divided cells are competent to receive the T-DNA.

Double immunohistological staining of VDI and β-glucuronidase (GUS) was conducted for Agrobacterium-transformed BY2 cells, using anti-VDI or anti-GUS as the primary antibody and anti-rabbit IgG conjugated-Cy3 or anti-mouse IgG conjugated-FITC as the secondary antibody. The samples were prepared after cocultivation of BY2 cells and preinduced A. tumefaciens for 3 days.

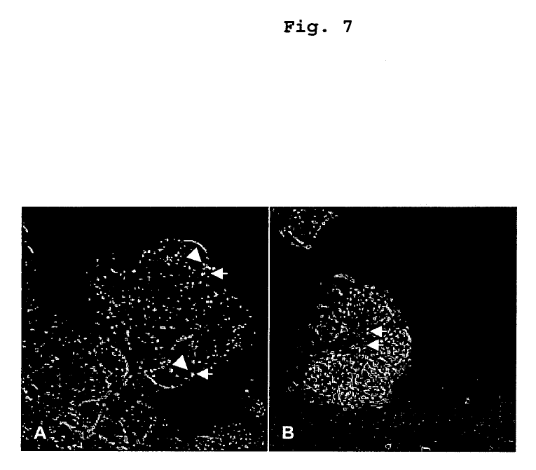

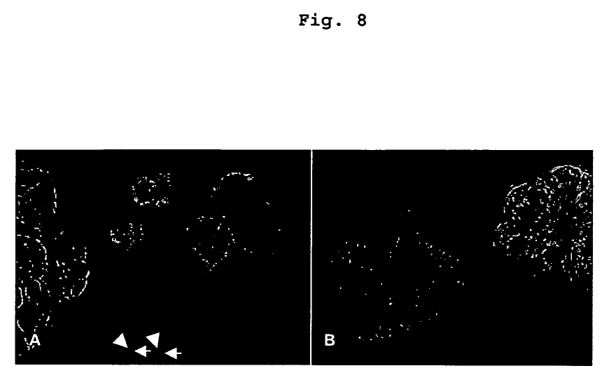

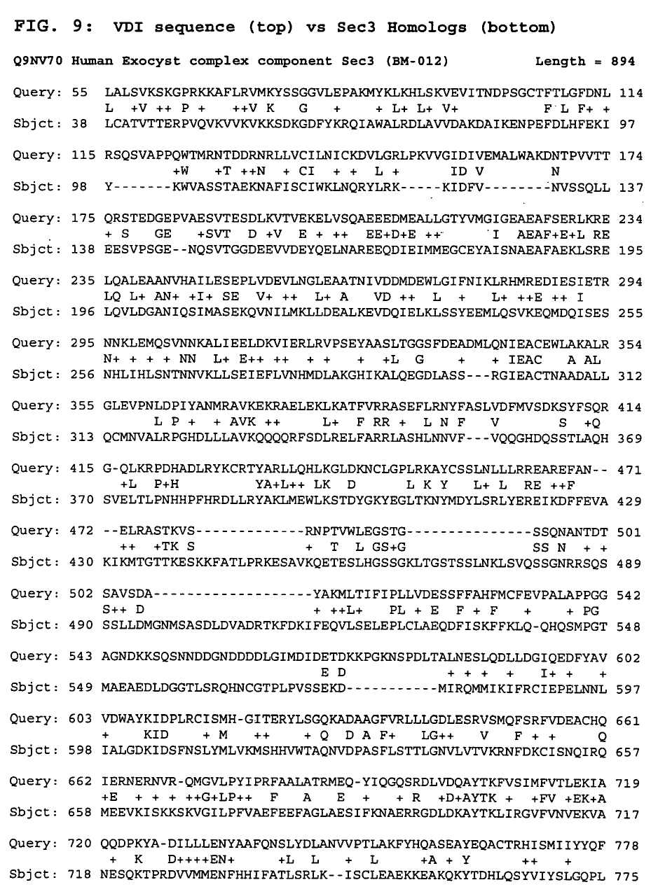

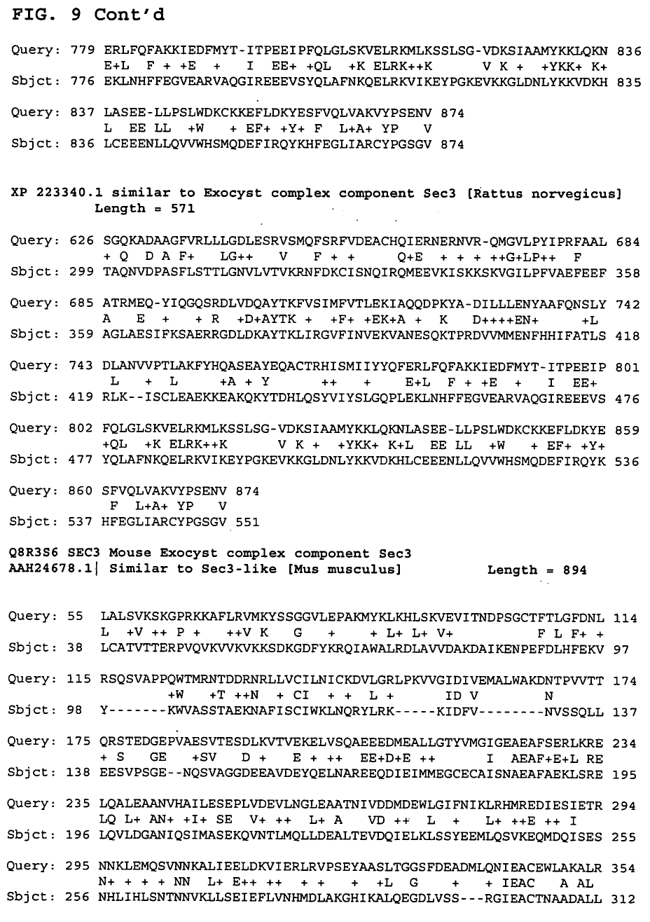

As shown in FIG. 7, the VDI (red dots) and reporter marker β-glucuronidase (GUS) (green dots) coexisted in the same transformed cells. The BY2 cells producing VDI also expressed the GUS protein encoded on the T-DNA delivered from Agrobacterium. In contrast, the remaining cells that lacked the VDI protein also did not produce any detectable β-glucuronidase protein. This suggests that VDI is associated with Agrobacterium-mediated transformation of plants.

It is likely that only cells in a certain stage of the cell cycle can produce VDI and consequently are competent to receive T-DNA and transformed by Agrobacterium. Cells in other stage(s) could not produce VDI and consequently are not competent to receive T-DNA. This is consistent with the observation that roughly half of undifferentiated plant cells did not take up any T-DNA.

Coexistence of VDI with T-DNA was observed in transformed BY2 cells. The samples were prepared after coincubation of BY2 cells and preinduced LBA4404(pIG121-Hm) for 1 day. They were then subjected to the VOID procedure to observe T-DNA, and immunohistology to localize VDI. The VOID procedure was performed with the same fragment of the GUS gene as the probe, which was labeled with the Biotin High Prime kit; anti-Avidin was used as the antibody to detect the T-DNA molecules (green dots in FIG. 8). Immunohistology was performed with anti-VDI and anti-rabbit IgG conjugated with Cy3 as the primary and secondary antibody, respectively.

As shown in FIG. 8, T-DNA molecules (green dots) appeared to coexist with VDI (red dots) together in the plant cells transformed by A. tumefaciens. In contrast, no T-DNA molecules were detectable in the cells that did not produce VDI. This demonstrates that VDI played an important role in the Agrobacterium-mediated plant transformation.

It was observed that VDI and T-DNA molecules were quite close to each other (FIG. 8); this suggests that VDI may assist trafficking of the T-complex in the plant cytoplasm. As shown in FIGS. 5, 7 and 8, VDI protein appeared to be randomly distributed in the cytoplasm of untransformed BY2 cells, while VDI was clustered with T-DNA in the transformed BY2 cells at 1 day of cocultivation. After cocultivation for 3 days, VDI protein became randomly distributed in the cytoplasm of plant cells like the untransformed cells. In the control experiment, VDI was still randomly distributed in the cytoplasm of BY2 cells, which were cocultivated with MX243 (virB mutant strain) that is unable to deliver T-DNA (Stachel, S. E., Nester, E. W. 1986. The genetic and transcriptional organization of the vir region of the A6 Ti plasmid of Agrobacterium tumefaciens. EMBO J., 5, 1445-1454) (FIG. 7B and FIG. 8B). These results clearly demonstrate that VDI actually participated in the process of Agrobacterium-mediated plant transformation.

The results described above indicate that VDI can be used as a molecular marker for cell competency to receive T-DNA. Thus identification of VDI in a cell indicates that the cell is transformation competent. Expression of cellular VDI can be determined using known methods in the art, including immuno detection with anti-VDI antibodies, such as Western blotting and ELISA, and immunoprecipitation of metabolically labeled cells using anti-VDI antibodies.

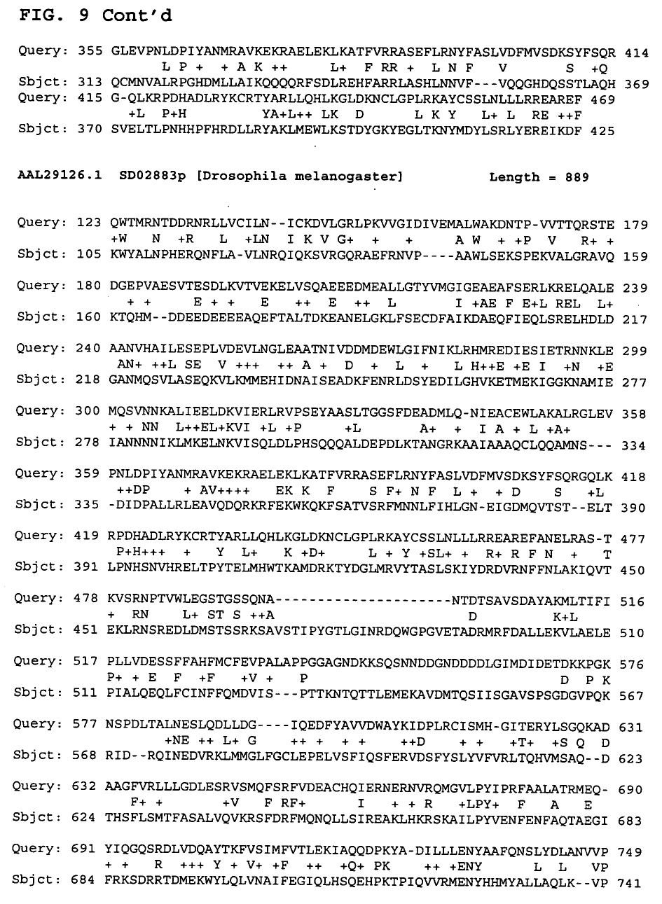

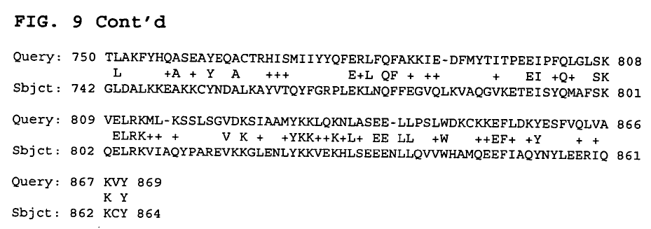

The plant VDI protein is homologous to the human Sec3 throughout the entire length of the proteins (see FIG. 9 for a comparison of VDI with a number of exemplary Sec3 sequences). This indicates that VDI is an ortholog of the human Sec3 protein. The Sec3 homologs are well conserved in eukaryotic cells. It is thus contemplated that homologs of Sec3, apart from VDI, also are useful as molecular markers for cell competency to receive exogenous nucleic acid. Examples of Sec3 homologs include human Sec3 (Accession number Q9NV70 and NP_{—}060731.1), Drosophila Sec3 (Accession number Q9VVG4), rat Sec3 (Accession number XP_{—}223340.1 and XP_{—}223339.1), mouse Sec3 (Accession number AAH24678.1), and Caenorhabditis elegans Sec3 (Accession number NP_{‘}508530.1).

Sec3 is a component of the Exocyst complex, which is well conserved in eukaryotic cells. Yeast and human Exocyst complexes have been identified (Matern et al. Proc Natl Acad Sci USA Aug. 14, 2001;98(17):9648-53). These Exocyst complexes exist as protein complexes consisting of several protein components. Since components of the Exocyst complex co-localize with Sec3 protein (Matern et al. Proc Natl Acad Sci USA Aug. 14, 2001;98(17):9648-53), it is contemplated that other components of the Exocyst complexes may also be used as molecular markers of a cell's competency to receive exogenous nucleic acid.

VIII. Identifying, Characterizing and Producing Cells Competent to Receive Exogenous Nucleic Acid

It is contemplated that fusions of Sec3, including VDI, with Green Fluorescent Protein (GFP), is useful to identify, characterize and produce cells competent to receive exogenous nucleic acid. It is further contemplated that regulated expression of exogenous Sec3 will produce cells more competent to receive exogenous nucleic acid.

Since the Sec3 homolog VDI is correlated with transformation competence, competent cells can be selected if one is able to select for cells expressing Sec3 without killing the cells in the process. Methods for selecting cells expressing a particular protein are known in the art. For example, Sec3 protein may be fused in frame with GFP or its variants, and the fusion protein stably expressed in the cell under the control of the native Sec3 promoter. Stable transformants expressing Sec3-GFP can be directly isolated by fluorescence activated cell sorting (FACS) using appropriate excitation wavelengths and emission detector. Techniques for making the fusion constructs, stably introducing the constructs into cells, and isolating and characterizing cells are routinely practised in the art. In fact, a Sec3 homolog has been fused with GFP; the fusion protein has been expressed and correctly localized in the cells (Matern et al. Proc Natl Acad Sci USA Aug. 14, 2001;98(17):9648-53).

A variety of GFP mutants are available which have distinct spectral properties, improved brightness and enhanced expression and folding in mammalian cells compared to the native GFP (Green Fluorescent Proteins, Chapter 2, pages 19 to 47, edited Sullivan and Kay, Academic Press, U.S. Pat. No. 5,625,048, U.S. Pat. No. 5,777,079, and U.S. Pat. No. 5,804,387).

It is further contemplated that expression of Sec3 in cells that do not express endogenous Sec3 will enhance the cell's competence to receive exogenous nucleic acid. As an example, Sec3 may be provided to a cell by way of an expression vector. The level of Sec3 may be regulated by placing the gene encoding Sec3 under control of high expression eukaryotic promoter/enhancers such as the CMV promoter/enhancer, SV40 promoter/enhancer, RSV LTR,herpes simplex thymidine kinase promoter. In a preferred embodiment, inducible promoters may be used to drive expression of Sec3 so that Sec3 expression can be turned on only when required, i.e. when nucleic acid delivery is carried out.

Inducible promoters contain transcription regulatory regions that function to transcribe mRNA only when inducing conditions are present. Examples of suitable inducible promoters include the E. coli lac operator responsive to IPTG, the metallothionein promoter metal-regulatory-elements responsive to heavy-metal (e.g. zinc) induction, the phage T7 lac promoter responsive to IPTG, the various heat-shock promoters, the mouse mammary tumor virus (MMTV) steroid-inducible promoter, the synthetic GAL4-VP16 inducible system, Stratagene's LacSwitch™ inducible mammalian expression system, glucocorticoid response element containing promoter, and the ectdysone promoter (U.S. Pat. No. 6,333,318).

### EXAMPLES

Agrobacterium-Mediated Transformation

Tobacco (Nicoliana tabacum) BY2 suspension-cultured cells were maintained in Murashige and Skoog (MS) liquid medium (Murashige, T., and Skoog, F. 1962. Physiol Plant 15: 473-497) supplemented with 0.2 mg/L of 2,4-D; the cultures were incubated at RT with shaking at 100 rpm and subcultured every week with a 4% inoculum. A. tumefaciens was grown overnight on AB medium; the cells were collected and then resuspended in IB medium (Cangelosi et al. 1991. Methods Enzymol., 204, 384-97) supplemented with 100 μM acetosyringone (AS). The cells were incubated at 28° C. for 16-18 hr. After washing with MS medium, 100 μl of the bacterial cell suspension (5×10⁸ cells/ml) was added to 4 ml of BY2 cell suspension that was 3 days old after the weekly subculturing. After incubation at RT for a certain time interval, the bacterial cells were washed away from the plant cells as described previously (Lee et al. 1999. J. Bacteriol. 181(1):186-196). The plant cells were then subjected to the GUS assay or VOID.

VOID Monitoring of T-DNA in Transit

BY2 cells were subjected to the following VOID procedure after A. tumefaciens had transferred T-DNA into the BY2 cells. The cells were fixed in 2% paraformaldehyde for 2 h and then were washed for 3 times with a freshly prepared fixative solution (ethanol mixed with glacial acetic acid at a ratio of 3:1). They were kept in the fixative solution at −20° C. until use.

The fixed cells were transferred to clean slides; the slides were allowed to air-dry for 1-2 days at RT. Immediately before in situ hybridization, the fixed cells on the slides were incubated in 0.2 % cellulase (in 0.01 M citrate buffer, pH 4.8) for 30 min at 37° C. After washing for 3 times in 0.01 M citrate buffer for 10 min, the cells were permeabilized with 0.2% Triton X-100 in PBS buffer for 10 min at 4° C. The cells were then washed for 3 times in PBS buffer for 10 min; they were treated with 100 μg/ml of RNase A in 2×SSC for 60 min at 37° C.

After washing 3 times in 2×SSC for 5 min, the slides were dehydrated in a 70%, 90% and 100% ethanol series. After denaturation in hybridization oven at 80° C. for 10 min, each slide was incubated with 20 μl of the hybridization mixture that had been heated at 75° C. for 10 min and then chilled on ice for 10 min. The hybridization mixture contained 50% deionized formamide, 10% dextran sulfate, 2×SSC, 0.01% salmon sperm DNA and 10 ng/μl of a DNA probe that had been labeled with digoxigenin-11-dUTP (DIG) using the DIG High Prime kit (Roche Diagnostics) with random primers and denatured at 95° C. for 10 min and chilled on ice for 10 min. The slides were then covered with a clean coverslip and incubated overnight at 37° C. in a humid chamber.

After the coverslips were removed, the slides were washed twice in a solution containing 50% formamide and 2×SSC for 15 min at 37° C., washed once at 37° C. in 2×SSC for 15 min, and then washed once at RT in 2×SSC for 15 min, and finally washed once at RT for 5 min with PBS buffer. The slides were incubated in a blocking solution (3% BSA in PBS buffer) at 37° C. for 1 hr. To each slide was added 100 μl of rhodamine-conjugated anti-DIG antibody (Roche Diagnostics) diluted (1:200) in the blocking solution. The slide was covered with a coverslip and incubated in a humid chamber at 37° C. for 45 min. After washing 4 times with 2×SSC containing 0.1% Tween-20 for 10 min, the slides were then dehydrated with a 70%, 90% and 100% ethanol series. The slides were finally air-dried and mounted with Vectashield mounting medium (Vector laboratories) containing PicoGreen (Molecular Probes) that can counterstain the nuclei of BY2 cells.

The slides were examined with an Olympus Fluoview 300 confocal microscope system. The excitation light for the green and red signal was 488 nm and 543 nm, respectively. The emission for the green and red signal was 515-560 nm bandpass filter and 565 nm longpass filter, respectively. The images for the green and red signals were overlapped in a computer by using the software provided by Olympus.

Use of VOID to Identify Molecular Markers Associated with Transformation

To determine the subcellular location of a plant protein VDI (which interacts with Agrobacterium protein VirD2), the plant cells were fixed in 2% paraformaldehyde in 1×PBS/pH 7.4 for 3 hrs at room temperature. After cells were affixed to slides pre-coated with poly-L-lysine (Sigma), the slides were washed 3 times in 1×PBS for 10 min. After the cells were digested with 0.2% cellulase in 0.01 M citrate buffer (pH 4.8) for 30 min at 37° C., they were permeabilized in 0.2% Triton X-100 (in PBS) for 5 min at 4° C. The cells were washed 3 times in PBS for 10 min and blocked in PBS containing 3% BSA for 1 hr. To each slide, 100 μl of primary antibody (anti-VDI) (diluted 1:100 into the blocking solution) was then added. The slides were covered with a coverslip and incubated for 1 hr at RT in a humid chamber. After being washed 3 times with PBS containing 3% BSA for 10 min, the slides were incubated in 100 μl of secondary antibody (FITC or CY3 conjugated) at a dilution of 1:100 with a coverslip for 1 hr in a humid chamber at RT. The slides were washed 3 times in PBS containing 0.1% Tween-20 for 10 min, then blown dry and mounted with a drop of Vectashield mounting medium (Vector Inc) with the coverslip sealed with clear nail polish to prevent drying and movement under the microscope. In some cases, the mounting medium was supplemented with PicoGreen (Molecular probes Inc) that can counterstain the nuclei of plant cells.

The foregoing is considered as illustrative only of the principles of the invention. Since numerous modifications and changes will readily occur to those skilled in the art, it is not desired to limit the invention to the exact modes of operation shown and described. Accordingly, all suitable modifications and equivalents may be resorted to, falling within the scope of the invention.

1. A process for monitoring exogenous nucleic acid in transit, the nucleic acid having been introduced into a cell, the process comprising:
(a) providing a biological sample containing cells into which exogenous nucleic acid has been introduced, wherein the exogenous nucleic acid is in transit;
(b) fixing the cells; and
(c) subjecting the cells to an in situ hybridization procedure which comprises contacting the permeabilized cells with a probe which hybridizes to the exogenous nucleic acid; and
(d) visualizing the exogenous nucleic acid in transit.

2. The process according to claim 1 for determining the number of exogenous nucleic acid in the cytoplasm or in the nucleus.

3. The process according to claim 1 for determining whether the exogenous nucleic acid is in the cytoplasm or the nucleus.

4. The process according to claim 1 for determining the length of time required for the exogenous nucleic acid to appear in the cytoplasm.

5. The process according to claim 1 for determining the length of time required for the exogenous nucleic acid to appear from the cytoplasm to the nucleus.

6. The process according to claim 1 for determining the efficiency of delivery of the nucleic acid into the nucleus, the process further comprising the step of measuring the ratio of the number of the exogenous nucleic acid in the nucleus to the number of the exogenous nucleic acid in the cytoplasm.

7. The process according to claim 1 for assessing risk associated with introduction of the exogenous nucleic acid into the cell, the process further comprising the step of determining the number of exogenous nucleic acid in the cytoplasm and in the nucleus at different time intervals after the exogenous nucleic acid has been introduced; determining the ratio of exogenous nucleic acid in the nucleus to cytoplasm at each interval; and predicting, in accordance with said ratio and number of exogenous nucleic acid introduced, the risk associated with introduction of the exogenous nucleic acid into the cell.

8. A process for determining the optimum parameters for obtaining a desired copy number of exogenous nucleic acid introduced into the cell, the process comprising:
(a) introducing an exogenous nucleic acid into a cell under a set of parameters;
(b) monitoring the exogenous nucleic acid according to the process defined in claim 1 to determine the number of exogenous nucleic acid in the cytoplasm or in the nucleus at different time intervals after the nucleic acid has been introduced; and
(c) determining the set of parameters under which the exogenous nucleic acid is delivered in the desired copy number into the cell.

9. The process according to claim 8 wherein one of the parameters is the length of time in which the exogenous nucleic acid is in contact with the cell.

10. The process according to claim 8 wherein one of the parameters is the ability of a gene delivery vector to deliver the exogenous nucleic acid.

11. A process for determining the proportion of cells competent to receive exogenous nucleic acid, the process comprising:
(a) introducing an exogenous nucleic acid to a portion of a population of cells;
(b) monitoring the exogenous nucleic acid according to the process defined in claim 1 to determine the presence of the exogenous nucleic acid in the cell; and
(c) determining the number of cells in which the exogenous nucleic acid is present as a proportion of the portion of cells, wherein the proportion is the proportion of cells of the population competent to receive the exogenous nucleic acid.

12. A process for identifying whether a cell contains an exogenous nucleic acid, wherein the exogenous nucleic acid is free of sequences encoding a selection marker or reporter protein intended to select for or identify the cell as containing the exogenous nucleic acid, the process comprising:
(a) introducing the exogenous nucleic acid into the cell; and
(b) monitoring the exogenous nucleic acid according to the process defined in claim 1;
wherein visualization of the nucleic acid in the cell indicates that the cell contains the exogenous nucleic acid.

13. A process for identifying a molecular marker associated with the competency of a cell to receive exogenous nucleic acid, wherein the cell comprises an antigen, the process comprising:
(a) introducing an exogenous nucleic acid to the cell;
(b) monitoring the exogenous nucleic acid according to the process defined in claim 1;
(c) testing the fixed cells for binding of the antigen with an antibody, wherein the antibody is capable of binding to the antigen in the fixed and permeabilized cell; and
(d) determining whether the antigen co-localizes with the exogenous nucleic acid in transit;
wherein co-localization of the exogenous nucleic acid in transit with the antigen indicates that the antigen is a molecular marker associated with transformation competency.

14. A process for identifying a cell that is competent for receiving exogenous nucleic acid, the process comprising monitoring the exogenous nucleic acid according to the process defined in claim 1 for presence of the exogenous nucleic acid in the cell.

15. The process according to any one of claims 1 to 14 wherein the nucleic acid is DNA.

16. The process according to claim 15 wherein the DNA is introduced into the cell by Agrobacterium.

17. The process according to claim 1 wherein the in situ hybridization procedure is fluorescence in situ hybridization.

18. The process according to claim 1 wherein the cell is a plant cell.

19. The process according to claim 18 further comprising the step of removing the cell wall.

20. The process according to claim 1, further comprising the step of permeabilizing the cells prior to contacting the cells with the probe.

21. A process for identifying a cell competent to receive exogenous nucleic acid, the process comprising the step of identifying expression of a Sec3 protein in the cell, wherein Sec3 expression indicates that the cell is competent to receive exogenous nucleic acid.

22. The process according to claim 21, wherein the Sec3 protein is VirD2-Interacting protein (VDI), and wherein the cell is a plant cell.

23. A process for identifying a cell competent to receive exogenous nucleic acid, comprising the step of identifying expression of a component of Exocyst complex in the cell, wherein expression of the component indicates that the cell is competent to receive exogenous nucleic acid.

24. The process according to 21 or 23 wherein the cell is a plant cell.

25. A kit for monitoring exogenous nucleic acid in transit, the nucleic acid having been introduced into a cell, the kit comprising:
(a) reagents for fixing the cells;
(b) reagents for permeabilizing the fixed cells;
(c) reagents for in situ hybridization of a probe with the exogenous nucleic acid; and
(d) instructions for using the reagents (a) to (c) to monitor the exogenous nucleic acid in transit.

26. A process for producing cells competent to receive exogenous nucleic acid, the process comprising the step of expressing Sec3 protein under control of an inducible promoter in the cell.

27. The process according to claim 26, wherein the Sec3 protein is VirD2-Interacting protein (VDI), and wherein the cell is a plant cell.
